(19) United States
(12) Patent Application Publication
(10) Pub. No.: US 2019/0038338 A1
(43) Pub. Date: Feb. 7, 2019

(54) Power Supply Device for High-Frequency Treatment Tool, High-Frequency Treatment System, and Method of Controlling High-Frequency Treatment Tool

(71) Applicant: Olympus Corporation, Tokyo (JP)

(72) Inventor: Tateyuki Sugawara, Kanagawa (JP)

(73) Assignee: Olympus Corporation, Tokyo (JP)

(21) Appl. No.: 16/158,355

(22) Filed: Oct. 12, 2018

Related U.S. Application Data
(63) Continuation of application No. PCT/JP2016/061819, filed on Apr. 12, 2016.

(51) Int. Cl. A61B 18/12; A61B 18/14; A61B 17/12

(52) U.S. Cl. CPC A61B 18/1206; A61B 2018/00708; A61B 17/1214; A61B 18/1485

(57) ABSTRACT

A power supply device used with a high-frequency treatment tool includes a power supply, a detection circuit, and a controller. The power supply supplies high-frequency electric power to an electrode of the high-frequency treatment tool. The detection circuit detects a first signal and a second signal. The first signal is a signal representing an output voltage output from the power supply to the high-frequency treatment tool. The second signal is a signal representing electric power returned from the high-frequency treatment tool back to the power supply. The controller includes one or more processors as hardware. The one or more processors calculate a return loss representing a state of contact between a living tissue and the electrode based on the first signal and the second signal. The one or more processors control the power supply to change its output between a first output level and a second output level.

### CROSS-REFERENCE TO RELATED APPLICATION

This application is a continuation application of PCT Application No. PCT/JP2016/061819 filed on Apr. 12, 2016, which is hereby incorporated by reference in its entirety.

### TECHNICAL FIELD

The disclosed technology relates to a power supply device for a high-frequency treatment tool, a high-frequency treatment system, and a method of controlling a high-frequency treatment tool.

### DESCRIPTION OF THE RELATED ART

Generally, there has been known a treatment system for treating a living tissue using high-frequency electric power. In such a treatment system, for example, an electric surgical knife is connected to one pole of a high-frequency power supply and a counter electrode plate is connected to the other pole thereof. The treatment system treats a living tissue when a high-frequency current output from the electric surgical knife is retrieved by the counter electrode plate. A high-frequency treatment tool using such a high-frequency current is used to make an incision in a living tissue or to stop bleeding from a living tissue.

It is important in a treatment system that outputs high-frequency electric power to grasp the output thereof and the state of a living tissue to be treated. For example, U.S. Pat. No. 6,019,757 discloses a technology relating to a system for occluding blood vessels or the like. This document discloses a technology for monitoring reflected electric power from an occluding device in order to detect the occlusion of a blood vessel. The document also reveals that the reflected electric power and a predetermined threshold value are compared with each other. Furthermore, U.S. Pat. No. 6,296,636 discloses a technology relating to controlling the output of a high-frequency treatment tool. This document reveals a technology for restraining an overcurrent and sparks that can occur when an electrode touches a low-impedance object.

Monopolar high-frequency treatment tools as described hereinbefore include a handpiece having an electrode and a counter electrode plate. With such a high-frequency treatment tool, when the surgeon presses a button switch disposed on a portion of a grip of the handpiece, for example, a power supply device outputs a preset level of electric power to the handpiece.

While such a high-frequency treatment tool is in use, the user may not necessarily turn on the output switch until when the electrode of the handpiece and a living tissue to be treated are held in contact with each other. For example, the user may turn on the output switch before or after the electrode of the handpiece and the living tissue contact each other.

Accordingly, there is a need to provide a power supply device for a high-frequency treatment tool, a high-frequency treatment system, and a method of controlling a high-frequency treatment tool, which are capable of grasping a situation that an electrode and a living tissue are in and restraining an unintended large electric charge.

### BRIEF SUMMARY OF EMBODIMENTS

It is an object of the disclosed technology to provide a power supply device for a high-frequency treatment tool, a high-frequency treatment system, and a method of controlling a high-frequency treatment tool, which are capable of grasping a situation that an electrode and a living tissue are in and restraining an unintended large electric charge.

According to an aspect of the disclosed technology, there is provided a power supply device used with a high-frequency treatment tool. The power supply device includes a power supply, a detection circuit, and a controller. The power supply supplies high-frequency electric power to an electrode of the high-frequency treatment tool. The detection circuit detects a first signal and a second signal. The first signal is a signal representing an output voltage output from the power supply to the high-frequency treatment tool. The second signal is a signal representing electric power returned from the high-frequency treatment tool back to the power supply. The controller includes one or more processors as hardware. The one or more processors calculate a return loss representing a state of contact between a living tissue and the electrode based on the first signal and the second signal. The one or more processors control the power supply to change its output between a first output level and a second output level. The first output level is an output level for treating the living tissue. The second output level is an output level lower than the first output level. The one or more processors set the output of the power supply to the second output level when the return loss meets a predetermined switching condition.

According to another aspect of the disclosed technology, a high-frequency treatment system includes the power supply device and the high-frequency treatment tool.

According to a further aspect of the disclosed technology, there is provided a method of controlling a high-frequency treatment tool of a high-frequency treatment system. The method includes supplying high-frequency electric power from a power supply of the high-frequency treatment system to an electrode of the high-frequency treatment tool. Next, the method includes detecting a first signal and a second signal. The first signal is a signal representing an output voltage output from the power supply to the high-frequency treatment tool. The second signal is a signal representing electric power returned from the high-frequency treatment tool back to the power supply. The method includes calculating a return loss representing a state of contact between a living tissue and the electrode based on the first signal and the second signal. The method includes controlling the power supply to change its output from a first output level to a second output level when the return loss meets a predetermined switching condition. The first output level is an output level for treating the living tissue. The second output level is an output level lower than the first output level.

According to the disclosed technology, there are thus provided a power supply device for a high-frequency treatment tool, a high-frequency treatment system, and a method of controlling a high-frequency treatment tool, which are capable of grasping a situation that an electrode and a living tissue are in and restraining an unintended large electric charge.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view depicting by way of example an appearance of a treatment system according to an embodiment.

FIG. 2 is a block diagram depicting a general configurational example of the treatment system according to the embodiment.

FIG. 3 is a diagram depicting an example of a circuit arrangement of a detection circuit.

FIG. 4 is a diagram depicting flows of signals in the treatment system according to the embodiment.

FIG. 5 is a graph illustrative of an outline of an example in which the value of a return loss changes over time that elapses as an electrode is moved closer to a living tissue.

FIG. 6A is a view illustrative of the manner in which the electrode is moved closer to the living tissue, the electrode and the living tissue being sufficiently spaced apart from each other.

FIG. 6B is a view illustrative of the manner in which the electrode is moved closer to the living tissue, the electrode and the living tissue being close to each other with an electric discharge occurring therebetween.

FIG. 6C is a view illustrative of the manner in which the electrode is moved closer to the living tissue, the electrode and the living tissue being held in contact with each other.

FIG. 7A is a flowchart of an example of operation of a power supply device according to a first embodiment.

FIG. 7B is a flowchart of the example of operation of the power supply device according to the first embodiment.

FIG. 8 is a graph illustrative of the way in which the maximum value of a return loss is updated.

FIG. 9 is a table illustrative of the way in which the maximum value of the return loss is updated.

FIG. 10 is a graph illustrative of an example in which the return loss changes over time and output levels change as the return loss changes over time.

FIG. 11 is a graph illustrative of another example in which output levels change over time.

FIG. 12 is a graph illustrative of still another example in which output levels change over time.

FIG. 13 is a graph illustrative of yet another example in which output levels change over time.

FIG. 14 is a graph illustrative of yet still another example in which output levels change over time.

FIG. 15 is a graph illustrative of a further example in which output levels change over time.

FIG. 16A is a flowchart of an example of operation of a power supply device according to a modification of the first embodiment.

FIG. 16B is a flowchart of the example of operation of the power supply device according to the modification of the first embodiment.

FIG. 17 is a graph illustrative of an outline of an example in which the value of a return loss changes over time that elapses as an electrode is moved away from a living tissue.

FIG. 18A is a view illustrative of the manner in which the electrode is moved away from the living tissue, the electrode and the living tissue being held in contact with each other.

FIG. 18B is a view illustrative of the manner in which the electrode is moved away from the living tissue, the electrode and the living tissue being close to each other with an electric discharge occurring therebetween.

FIG. 18C is a view illustrative of the manner in which the electrode is moved away from the living tissue, the electrode and the living tissue being sufficiently spaced apart from each other.

FIG. 19A is a flowchart of an example of operation of a power supply device according to a second embodiment.

FIG. 19B is a flowchart of the example of operation of the power supply device according to the second embodiment.

FIG. 20 is a graph illustrative of the way in which the minimum value of a return loss is updated.

FIG. 21 is a table illustrative of the way in which the minimum value of the return loss is updated.

FIG. 22 is a graph illustrative of an example in which the return loss changes over time and output levels change as the return loss changes over time.

FIG. 23A is a flowchart of an example of operation of a power supply device according to a modification of the second embodiment.

FIG. 23B is a flowchart of the example of operation of the power supply device according to the modification of the second embodiment.

FIG. 24 is a flowchart of an example of operation of a power supply device according to a third embodiment.

FIG. 25 is a view depicting by way of example an appearance of a treatment system according to a modification.

FIG. 26 is a block diagram depicting a general configuration of the treatment system according to the modification.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

System Arrangement

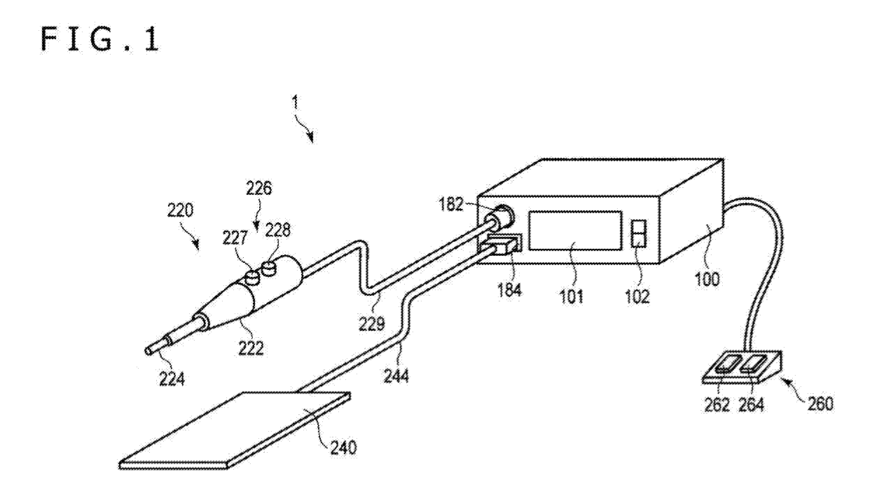

A first embodiment of the present invention will be described hereinafter with reference to the drawings. FIG. 1 is a view depicting by way of example an appearance of a treatment system 1 according to the present embodiment. As depicted in FIG. 1, the treatment system 1 includes a power supply device 100, a treatment tool 220, a counter electrode plate 240, and a foot switch assembly 260.

The treatment tool 220 is connected to an end of a first cable 229 that interconnects the treatment tool 220 and the power supply device 100 to one another. The other end of the first cable 229 is connected to a treatment tool terminal 182 of the power supply device 100. The treatment tool 220 includes a manipulator 222 and a distal-end electrode 224. The manipulator 222 is gripped by the user for manipulating the treatment tool 220. The distal-end electrode 224 is disposed on the distal end of the manipulator 222. The distal-end electrode 224 is applied to a living tissue to be treated when the treatment tool 220 treats the living tissue.

The manipulator 222 has a hand switch assembly 226 including a first switch 227 and a second switch 228. The first switch 227 is a switch for entering an input for controlling the power supply device 100 to produce an output in an incision mode. The incision mode is a mode for supplying a relatively high level of electric power to cauterize a living tissue to be treated which is contacted by the distal-end electrode 224. The second switch 228 is a switch for entering an input for controlling the power supply device 100 to produce an output in a hemostatic mode. The hemostatic mode is a mode for supplying a lower level of electric power than in the incision mode to cauterize a living tissue to be treated which is contacted by the distal-end electrode 224 and to modify the end face of the living tissue to stop bleeding therefrom. The foot switch assembly 260 includes a first switch 262 and a second switch 264. The first switch 262 of the foot switch assembly 260 has the same function as the first switch 227 on the treatment tool 220. The second switch 264 of the foot switch assembly 260 has the same function as the second switch 228 on the treatment tool 220. The user can thus selectively turn on and off the output of the treatment tool 220 using the first switch 227 and the second switch 228 on the treatment tool 220 or using the first switch 262 and the second switch 264 of the foot switch assembly 260.

The counter electrode plate 240 can be applied to the surface of the body of a patient to be treated. The counter electrode plate 240 is connected to an end of a second cable 244 that interconnects the counter electrode plate 240 and the power supply device 100. The other end of the second cable 244 is connected to a counter electrode plate terminal 184 of the power supply device 100. The power supply device 100 is a power supply for supplying electric power between the treatment tool 220 and the counter electrode plate 240. The power supply device 100 has a display panel 101 and a switch 102. The display panel 101 displays various items of information relating to the state of the power supply device 100. The user enters an output setting value such as for output electric power, a setting value for determining a cutting quality referred to as effect, and so on, for example, into the power supply device 100 using the switch 102.

When the treatment system 1 is in use, the user, who is generally a surgeon or the like, brings the distal-end electrode 224 into contact with a region to be treated, while pressing the first switch 227 or the second switch 228 on the treatment tool 220. At this time, a current output from the power supply device 100 flows between the distal-end electrode 224 and the counter electrode plate 240. As a result, a living tissue that is contacted by the distal-end electrode 224 is incised or stops bleeding.

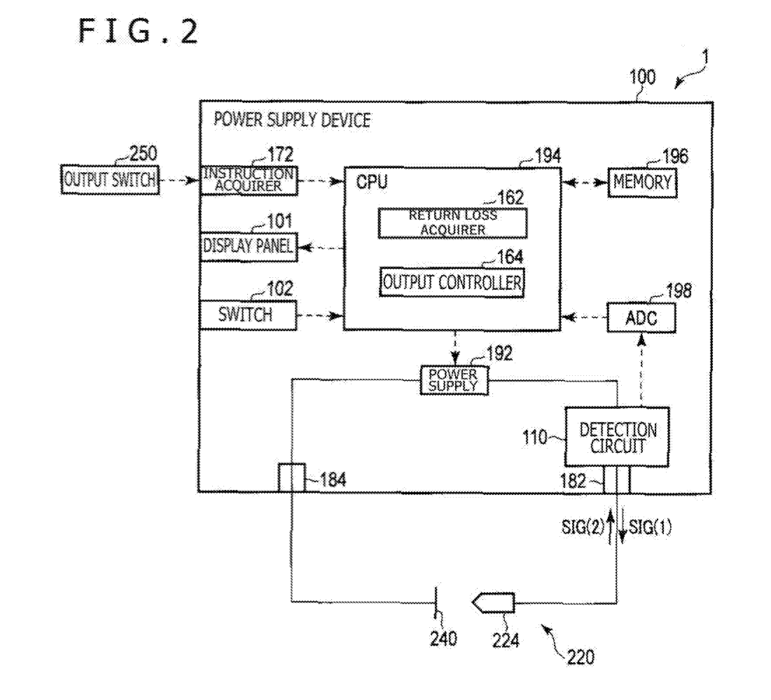

FIG. 2 depicts a general configuration of the treatment system 1. The power supply device 100 includes a power supply 192, a central processing unit (CPU) 194, a memory 196, and an analog/digital converter (ADC) 198. The CPU 194 controls various parts of the power supply device 100 and performs various arithmetic and processing operations. The CPU 194 thus functions as an arithmetic and processing unit. The memory 196 stores programs and various parameters required for the CPU 194 to operate. The functions of the CPU 194 may be performed by an integrated circuit such as an application specific integrated circuit (ASIC), a field programmable gate array (FPGA), or the like. Alternatively, the functions of the CPU 194 may be performed by a combination of integrated circuits including either one of a CPU, an ASIC, an FPGA, and the like. The ADC 198 converts analog signals output from a detection circuit 110, to be described hereinafter, into digital signals, and transmits the digital signals to the CPU 194. The power supply 192 acquires electric power from outside of the power supply device 100, and outputs alternating-current electric power according to processed results from the CPU 194.

The display panel 101 and the switch 102 referred to hereinbefore are connected to the CPU 194, which controls display operation of the display panel 101. The CPU 194 acquires information input to the switch 102 and reflects the acquired information for the control of the power supply device 100. The power supply device 100 also includes an instruction acquirer 172 including an analog/digital converter. Assuming that the hand switch assembly 226 or the foot switch assembly 260 is represented as an output switch 250, the instruction acquirer 172 acquires information input to the output switch 250 and transmits the acquired information to the CPU 194.

A detection circuit 110 is disposed in the vicinity of a treatment tool terminal 182 to which the treatment tool 220 of the power supply device 100 is connected. The detection circuit 110 detects a first signal (indicated as SIG(1)) and a second signal (indicated as SIG(2)). The first signal (SIG(1)) is a signal relating to output electric power, or first electric power, output from the treatment tool terminal 182 of the power supply device 100 to the treatment tool 220. The second signal (SIG(2)) is a signal relating to returning electric power, or second electric power, output from the treatment tool terminal 182 to the treatment tool 220 and returning from the treatment tool 220 to the treatment tool terminal 182. The first signal (SIG(1)) and the second signal (SIG(2)) are transmitted via the ADC 198 to the CPU 194. The first signal (SIG(1)) and the second signal (SIG(2)) may be amplified when necessary.

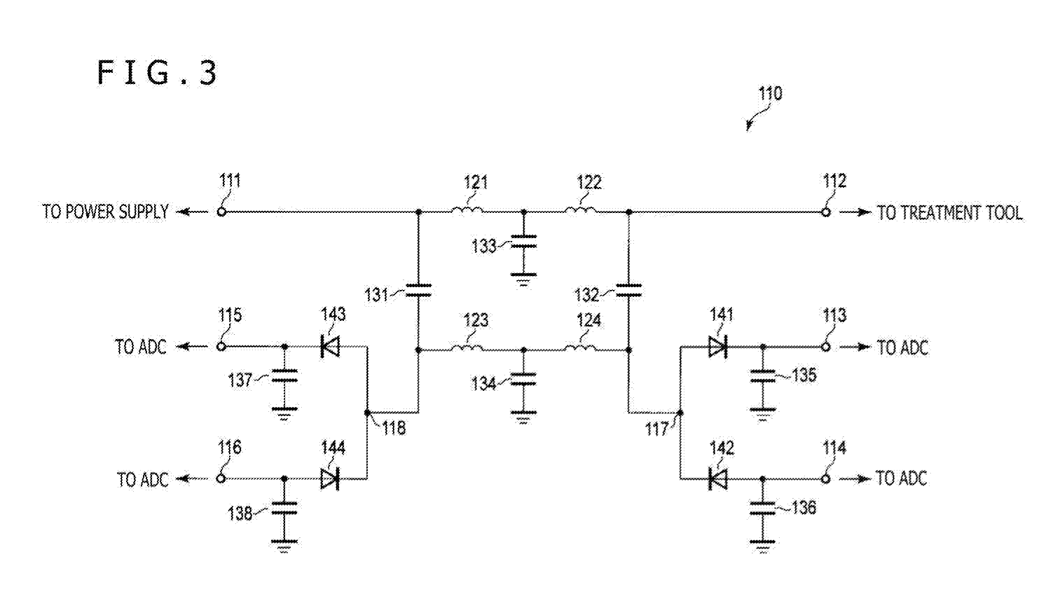

FIG. 3 depicts by way of example a circuit arrangement of the detection circuit 110. The detection circuit 110 includes coils, capacitors, and diodes.

Of the terminals of the detection circuit 110, a terminal to which a current output from the power supply 192 is input is referred to as a first terminal 111. Of the terminals of the detection circuit 110, a terminal connected to the treatment tool terminal 182 is referred to as a second terminal 112. The detection circuit 110 has two terminals for extracting the first signal (SIG(1)), and one of the terminals is referred to as a third terminal 113 and the other as a fourth terminal 114. The detection circuit 110 has two terminals for extracting the second signal (SIG(2)), and one of the terminals is referred to as a fifth terminal 115 and the other as a sixth terminal 116.

A first coil 121 and a second coil 122 are connected in series with each other between the first terminal 111 and the second terminal 112. The first coil 121 has an end connected to the first terminal 111, which end is connected to an end of a first capacitor 131. The other end of the first capacitor 131 is referred to as a second signal end 118. The second coil 122 has an end connected to the second terminal 112, which end is connected to an end of a second capacitor 132. The other end of the second capacitor 132 is referred to as a first signal end 117. A third capacitor 133 has an end connected between the first coil 121 and the second coil 122. The other end of the third connected 133 is connected to ground. A third coil 123 and a fourth coil 124 are connected in series with each other between the first signal end 117 and the second signal end 118. A fourth capacitor 134 has an end connected between the third coil 123 and the fourth coil 124. The other end of the fourth capacitor 134 is connected to ground.

A first diode 141 has an anode connected to the first signal end 117. A cathode of the first diode 141 is connected to the third terminal 113. A fifth capacitor 135 has an end connected to the cathode of the first diode 141. The other end of the fifth capacitor 135 is connected to ground. The fifth capacitor 135 serves to perform charge-to-voltage conversion, allowing a signal voltage to be extracted from the third terminal 113.

A second diode 142 has a cathode connected to the first signal end 117. An anode of the second diode 142 is connected to the fourth terminal 114. A sixth capacitor 136 has an end connected to the anode of the second diode 142. The other end of the sixth capacitor 136 is connected to ground. The sixth capacitor 136 serves to perform charge-to-voltage conversion, allowing a signal voltage to be extracted from the fourth terminal 114.

A third diode 143 has an anode connected to the second signal end 118. A cathode of the third diode 143 is connected to the fifth terminal 115. A seventh capacitor 137 has an end connected to the cathode of the third diode 143. The other end of the seventh capacitor 137 is connected to ground. The seventh capacitor 137 serves to perform charge-to-voltage conversion, allowing a signal voltage to be extracted from the fifth terminal 115. A fourth diode 144 has a cathode connected to the second signal end 118. An anode of the fourth diode 144 is connected to the sixth terminal 116. An eighth capacitor 138 has an end connected to the anode of the fourth diode 144. The other end of the eighth capacitor 138 is connected to ground. The eighth capacitor 138 serves to perform charge-to-voltage conversion, allowing a signal voltage to be extracted from the sixth terminal 116.

The first terminal 111 and the second terminal 112 are thus arranged in a configuration symmetric to each other. The first signal end 117 and the second signal end 118 are arranged in a configuration symmetric to each other. The circuit arrangement depicted in FIG. 3 is in accordance with one embodiment, and the detection circuit 110 is not limited to the illustrated circuit arrangement, but may be of an asymmetrical circuit arrangement based on the illustrated circuit arrangement. With this circuit arrangement, the magnitude of a positive signal among signals correlated to a signal passing from the first terminal 111 to the second terminal 112 is acquired from the third terminal 113. The magnitude of a negative signal among the signals correlated to the signal passing from the first terminal 111 to the second terminal 112 is acquired from the fourth terminal 114. Similarly, the magnitude of a positive signal among signals correlated to a signal passing from the second terminal 112 to the first terminal 111 is acquired from the fifth terminal 115. The magnitude of a negative signal among the signals correlated to the signal passing from the second terminal 112 to the first terminal 111 is acquired from the sixth terminal 116.

The connected relation in FIG. 2 of the terminals depicted in FIG. 3 is as follows: The first terminal 111 is connected to the power supply 192. The second terminal 112 thereof is connected to the treatment tool 220 through the treatment tool terminal 182. The third and fourth terminals 113 and 114 thereof are connected to the ADC 198. The fifth and sixth terminals 115 and 116 thereof are also connected to the ADC 198.

The detection circuit 110 serves to acquire signals correlated to the signal (main signal) passing through the path between the first terminal 111 and the second terminal 112 from the third terminal 113, the fourth terminal 114, the fifth terminal 115 and the sixth terminal 116. The signals acquired from the third terminal 113, the fourth terminal 114, the fifth terminal 115 and the sixth terminal 116 are generally smaller than the main signals. The signals that are detected represent electric power. The electric power is converted into an analog voltage signal between the first signal end 117 and the third terminal 113 or the fourth terminal 114. Similarly, the electric power is converted into an analog voltage signal between the second signal end 118 and the fifth terminal 115 or the sixth terminal 116. These analog voltage signals are converted into digital signals by the ADC 198.

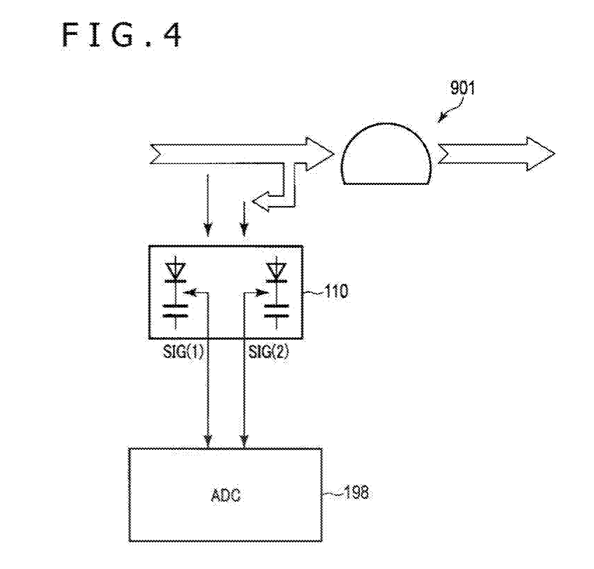

FIG. 4 schematically depicts electric power that passes through a patient 901 to be treated and signals obtained therefrom. As indicated by the outlined arrows in FIG. 4, electric power is supplied to the patient 901. Most of the electric power passes through the patient 901, whereas part of the electric power is reflected thereby. The detection circuit 110 acquires a signal depending on the electric power input to the patient 901 as the first signal (SIG(1)), which is transmitted to the ADC 198. The detection circuit 110 acquires a signal depending on the electric power having returned from the patient 901 as the second signal (SIG(2)), which is transmitted to the ADC 198. Information obtained based on the first signal (SIG(1)) and the second signal (SIG(2)) will be described hereinafter. It is assumed that the ratio of the second signal (SIG(2)) to the first signal (SIG(1)) is referred to as a return loss (indicated by RL). The return loss RL is expressed as:

RL=SIG(2)/SIG(1)

The return loss RL represents how much electric power output from the power supply device 100 is returned from a side of the treatment tool 220, i.e., represents a return ratio. When the return loss RL is large, for example, it is considered that there is a space between the distal-end electrode 224 and the living tissue, with no current flowing to the living tissue. The state between the distal-end electrode 224 and the living tissue, including the distance between the distal-end electrode 224 and the living tissue, can thus be presumed based on the first signal (SIG(1)) and the second signal (SIG(2)).

The CPU 194 calculates a return loss RL based on the first signal (SIG(1)) and the second signal (SIG(2)). The CPU 194 then controls the output of the power supply 192 using the calculated return loss RL. As described hereinbefore, the CPU 194 acquires the first signal (SIG(1)) and the second signal (SIG(2)) from the detection circuit 110. The CPU 194 performs a function as a return loss acquirer 162 for calculating a return loss RL. The CPU 194 also performs a function as an output controller 164 for controlling the output of the power supply 192. It is understood that when the distal-end electrode 224 and a living tissue 900 are somewhat spaced apart from each other, the output value of the high-frequency treatment tool can instantaneously deviate largely from a target value. In order to prevent the output value from instantaneously deviating largely from the target value, the treatment system 1 reduces the output of the power supply 192 when the return loss RL representing the state between the distal-end electrode 224 and the living tissue 900 satisfies a predetermined condition.

System Operation

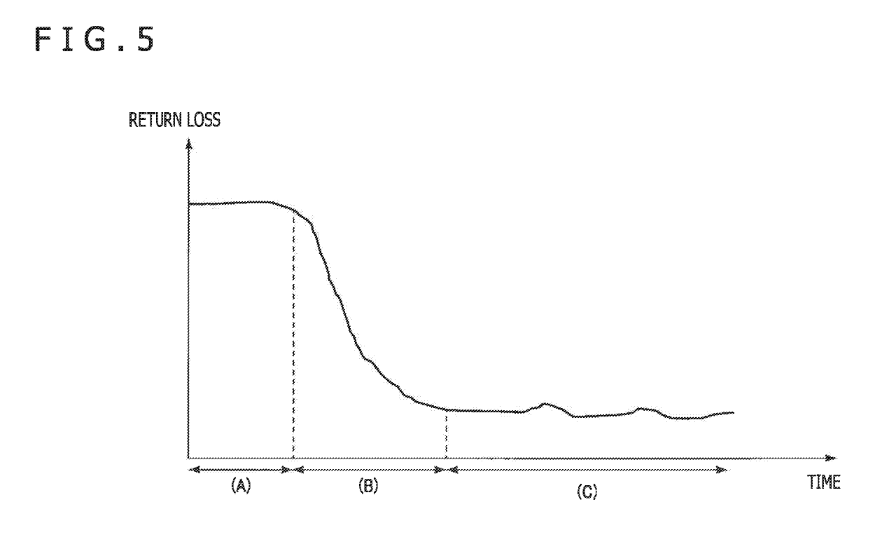

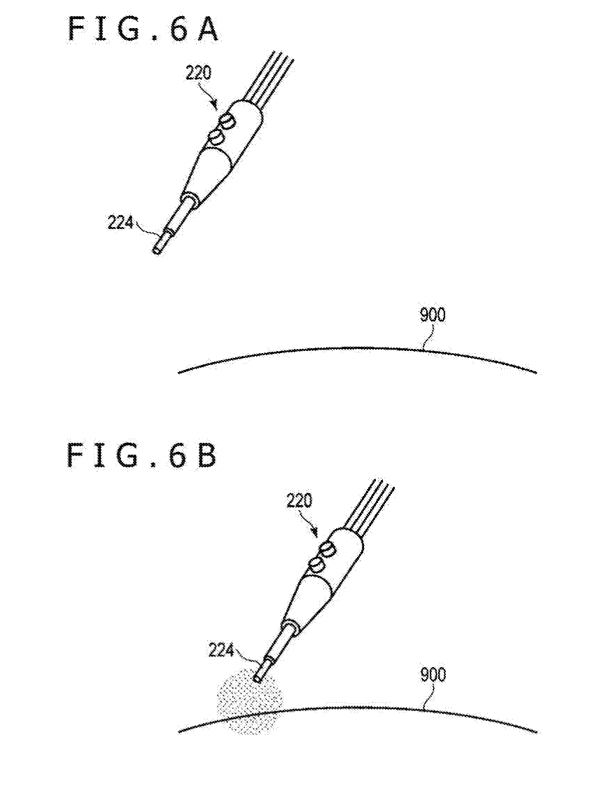

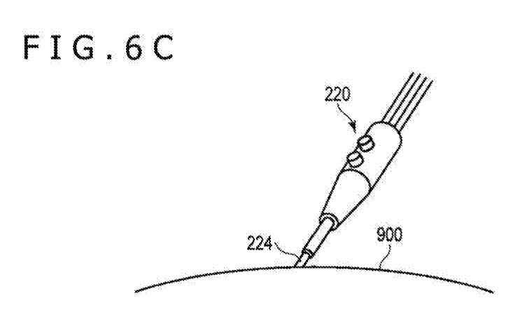

According to the first embodiment, the treatment system 1 operates to stop the output temporarily when the distance between the distal-end electrode 224 and the living tissue to be treated reaches a predetermined distance while the distal-end electrode 224 is moving closer to the living tissue. An outline of operation of the power supply device 100 according to the present embodiment will be described hereinafter with reference to FIGS. 5, 6A, 6B, and 6C. FIG. 5 has a horizontal axis representing time and a vertical axis representing the return loss RL calculated by the return loss acquirer 162. FIG. 5 illustrates the relation between time and the return loss RL as the distal-end electrode 224 moves gradually closer to the living tissue to be treated until the distal-end electrode 224 contacts the living tissue while a high-frequency voltage is being applied between the distal-end electrode 224 and the counter electrode plate 240. During a period indicated by (A) in FIG. 5, there is a sufficient distance between the living tissue 900 and the distal-end electrode 224, as depicted in FIG. 6A. At this time, the return loss RL is of a maximum value. During a period indicated by (B) in FIG. 5, the distal-end electrode 224 approaches the living tissue 900, bringing about an electric discharge between the distal-end electrode 224 and the living tissue 900. The electric discharge causes the return loss RL that is acquired to be lower than the return loss RL acquired during the period (A) in FIG. 5. FIG. 6B schematically illustrates the situation at a time included in the period indicated by (B) in FIG. 5, where an electric discharge is recognized in a region, depicted stipulated in FIG. 6B, between the distal-end electrode 224 and the living tissue 900. During a period indicated by (C) in FIG. 5, the distal-end electrode 224 is in contact with the living tissue 900, as depicted in FIG. 6C. During this period, the return loss RL is of a relatively small value.

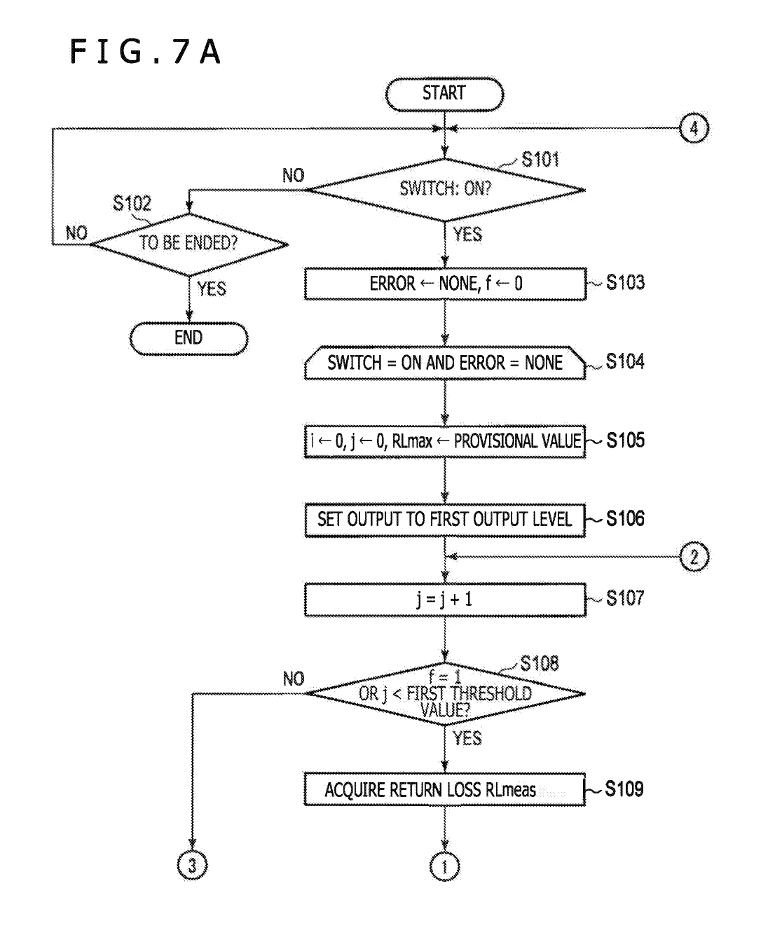

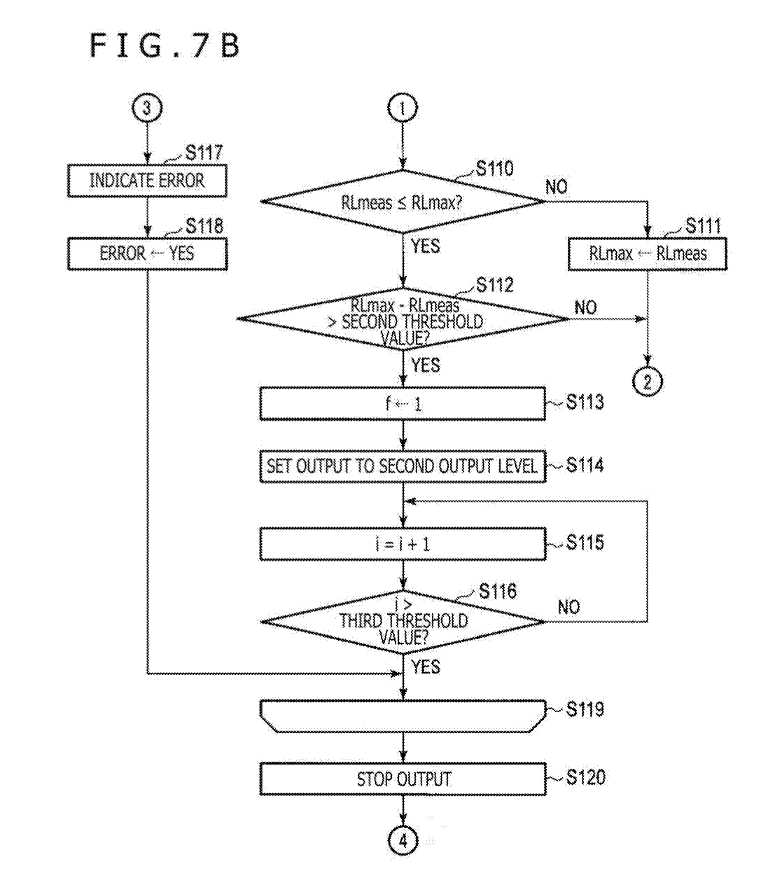

It is understood that during a part of the period indicated by (B) in FIG. 5, i.e., during a partial period while an electric discharge is occurring between the distal-end electrode 224 and the living tissue 900, the control process tends to become unstable due, for example, to an unintended excessive output current that flows. According to the present embodiment, the output is stopped during a part of the period indicated by (B) in FIG. 5. Operation of the power supply device 100 according to the present embodiment will be described hereinafter with reference to a flowchart depicted in FIGS. 7A and 7B. The present processing sequence is carried out when the power supply device 100 has its main power supply turned on.

In step S101, the output controller 164 determines whether or not the output switch 250, which represents the foot switch assembly 260 or the hand switch assembly 226 for giving a command to turn on or off the output, is ON. If the output switch 250 is not ON, then control goes to step S102. In step S102, the output controller 164 determines whether or not the present processing sequence is to be ended, e.g., whether or not the main power supply is turned off. If the present processing sequence is to be ended, then the present processing sequence is ended. If the present processing sequence is not to be ended, then control goes back to step S101. In other words, as long as the output switch 250 is OFF, control waits by repeating steps S101 and S102. If it is determined in step S101 that the output switch 250 is ON, then control goes to step S103. In step S103, the output controller 164 sets information relating to an error or no error to “NONE”, and sets a determination flag f to 0. The information relating to an error or no error and the value of the determination flag f are stored in the memory 196.

The processing from step S104 to step S119 is an iteration that is carried out under the condition that the output switch 250 is ON and there is no error. If the output switch 250 is OFF or there is an error, then the iteration is bypassed, and control goes to step S120.

In step S105, the output controller 164 initializes variables stored in the memory 196. Specifically, the output controller 164 sets a first counter i for measuring a blanking period, to be described hereinafter, to 0, and sets a second counter j for measuring a run-time error, to be described hereinafter, to 0. The output controller 164 also sets a maximum value RLₘₐₓ of the return loss RL, to be described hereinafter, to a provisional value. It is desirable that the provisional value should be of a value sufficiently smaller than a value expected to be the maximum value RLₘₐₓ. In step S106, the output controller 164 sets the output level of the power supply 192 to a first output level. The first output level is an output level, set by the user, for example, that is required for a treatment. The output level may be controlled by a voltage control process, a current control process, or other processes.

In step S107, the output controller 164 increments the value of the second counter j stored in the memory 196. In step S108, the output controller 164 determines whether or not the determination flag f is 1 or the second counter j is smaller than a predetermined first threshold value. If the determination flag f is 1 or the second counter j is smaller than the first threshold value, then control goes to step S109. In step S109, the output controller 164 acquires the return loss RL as a measured return loss RLₘₑₐₛ based on voltage values acquired by the detection circuit 110.

In step S110, the output controller 164 determines whether or not the acquired return loss RLₘₑₐₛ is equal to or smaller than the maximum value RLₘₐₓ of the return loss RL stored in the memory 196 at the time. If the return loss RLₘₑₐₛ is not equal to or smaller than the maximum value RLₘₐₓ, then control goes to step S111.

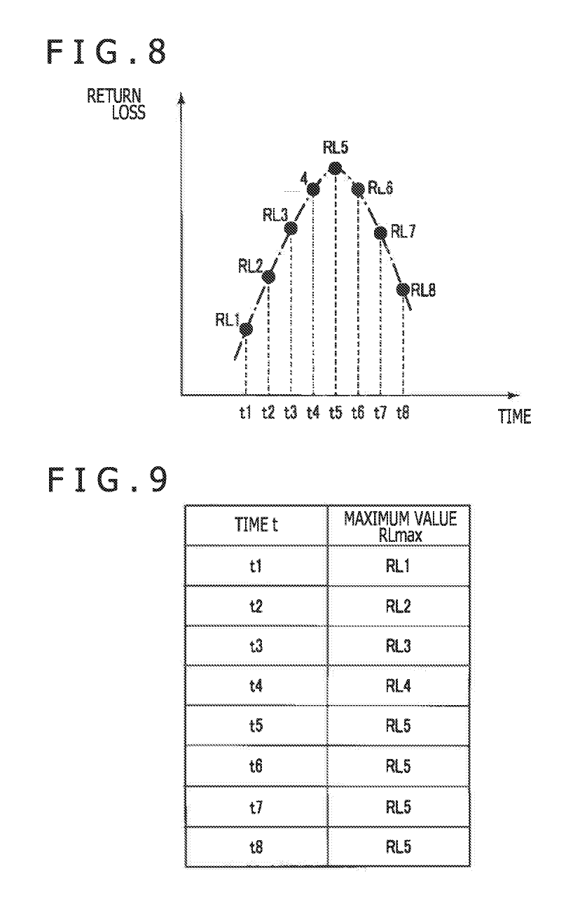

In step S111, the output controller 164 sets the maximum value RLₘₐₓ to the value of the acquired return loss RLₘₑₐₛ. In other words, the maximum value RLₘₐₓ is updated. Since the return loss RLₘₑₐₛ can be monotonously increased or reduced, rather than being monotonously reduced, the maximum value RLₘₐₓ of the return loss RL is updated as by the processing of step S111. For example, as depicted in FIG. 8, it is assumed that as time t elapses to t1, t2, t3, t4, and t5, the return loss RL gradually increases to RL1, RL2, RL3, RL4, and RL5. At this time, as depicted in FIG. 9, the maximum value RLₘₐₓ of the return loss RL gradually increases to RL1, RL2, RL3, RL4, and RL5. After the processing of step S111, control returns to step S107. If it is determined in step S110 that the return loss RLₘₑₐₛ is equal to or smaller than the maximum value RLmax, then control goes to step S112. For example, as depicted in FIG. 8, it is assumed that as time t elapses to t5, t6, t7, and t8, the return loss RLₘₑₐₛ gradually decreases to RL5, RL6, RL7, and RL8. At this time, as depicted in FIG. 9, the maximum value RLₘₐₓ of the return loss RL remains to be RL5, and is not updated.

In step S112, the output controller 164 determines whether or not the difference RLₘₐₓ−RLₘₑₐₛ, obtained by subtracting the return loss RLₘₑₐₛ from the maximum value RLₘₐₓ of the return loss RL, is larger than a predetermined second threshold value. If the difference RLₘₐₓ−RLₘₑₐₛ is not larger than the second threshold value, then control returns to step S107.

When the user brings the distal-end electrode 224 gradually closer to the living tissue 900, for example, the maximum value RLₘₐₓ is not changed, and the difference RLₘₐₓ−RLₘₑₐₛ becomes gradually larger.

If the determination flag f is not 1 and the second counter j is equal to or larger than the first threshold value in step S108, then control goes to step S117. In other words, if control has not proceeded to the processing of steps S113 through S116 and the value counted in step S107 is equal to or larger than the first threshold value, then control goes to step S117. This situation occurs when the user does not move the distal-end electrode 224 closer to the living tissue 900, as depicted in FIG. 6A, i.e., the user does not move the distal-end electrode 224 closer to the living tissue 900 while the output switch 250 is ON for longer than a predetermined period.

In step S117, the output controller 164 indicates an error representing that the user is not moving the distal-end electrode 224 closer to the living tissue 900. The output controller 164 may indicate the error by displaying it on the display panel 101, for example, or by outputting an alarm sound from a speaker, not depicted. In step S118, the output controller 164 sets the information relating to an error or no error to “YES”. Thereafter, control goes to step S119. Since the information relating to an error or no error has been set to “YES” at this time, the iteration of steps 104 through 119 is finished, and control goes to step S120.

If it is determined in step S112 that the difference RLₘₐₓ−RLₘₑₐₛ is larger than the second threshold value, then control goes to step S113. The condition that the difference RLₘₐₓ−RLₘₑₐₛ is larger than the second threshold value corresponds to a switching condition that is a condition for shifting to a restrained state where the output is reduced. In step S113, the output controller 164 sets the determination flag f stored in the memory 196 to 1. This determination flag indicates that the processing on and subsequent to step S113 is being carried out, i.e., the distal-end electrode 224 and the living tissue 900 are becoming closer to each other, as depicted in FIG. 6B. In step S114, the output controller 164 sets the output level of the power supply 192 to a second output level. Though the second output level will be described as zero, it may not be zero. If the output is zero, the output controller 164 stops the output of the power supply 192. When the switching condition is satisfied, the output is thus reduced.

In step S115, the output controller 164 increments the first counter i stored in the memory 196. In step S116, the output controller 164 determines whether or not the first counter i is larger than a predetermined third threshold value. If the first counter i is not larger than the third threshold value, then control goes back to step S115. In other words, the processing of steps S115 and S116 is repeated until the first counter i exceeds the third threshold value. Stated otherwise, control waits for a predetermined period. The period obtained by the first counter i, i.e., the period during which the output is stopped, will be referred to as a blanking period. The blanking period is ten milliseconds, for example. When the return loss satisfies a given switching condition, the treatment system 1 enters a restrained state where the output is reduced for a predetermined period. In other words, the state in which the output level is the second output level during the blanking period corresponds to the restrained state to which the treatment system 1 is shifted when the return loss satisfies the given switching condition. If it is determined in step S116 that the first counter i is larger than the third threshold value, then control goes to step S119. If the switch is ON and there is no error, then the processing from step S104 is repeated.

After the period indicated by (B) in FIG. 5 has elapsed, the processing that begins with step S104 is carried out again. In step S106, the output level of the power supply 192 is set again to the first output level. Since the maximum value RLₘₐₓ of the return loss RL has been set again to the provisional value in step S105, the difference RLₘₐₓ−RLₘₑₐₛ between the maximum value RLₘₐₓ and the return loss RLₘₑₐₛ does not become larger than the second threshold value, and the determination flag f is 1. Therefore, the processing of steps S107 through S112 is repeated. In other words, as long the switch is ON, the output at the first output level continues. If the switch is OFF or there is an error, then control goes to step S120. In step S120, the output controller 164 stops the output of the power supply 192. Thereafter, control returns to step S101.

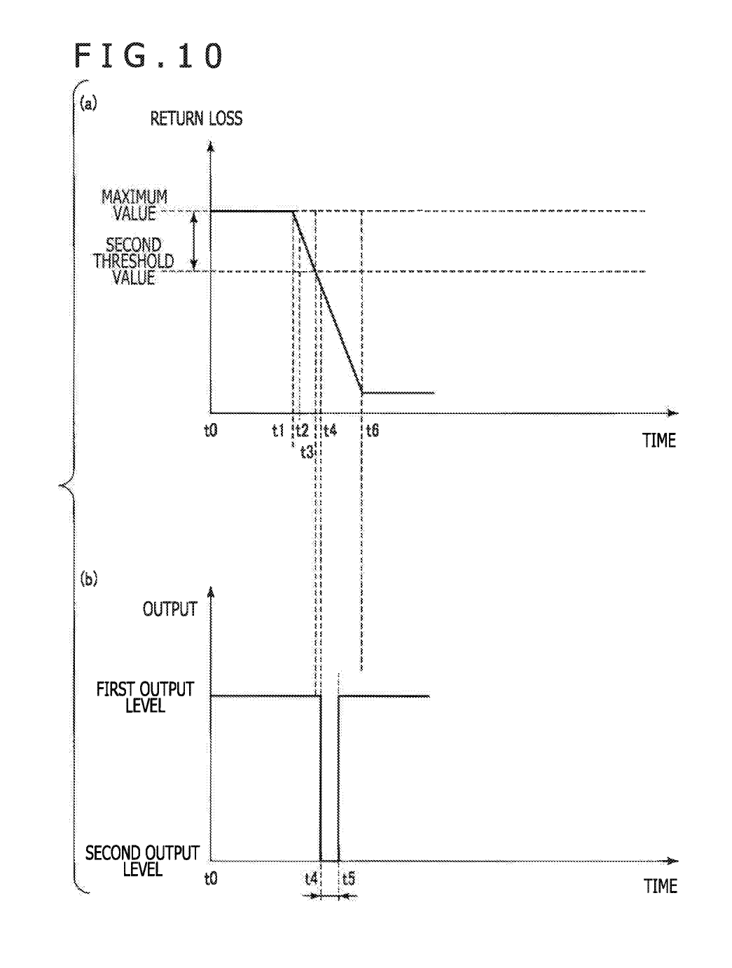

Time-dependent changes of the acquired return loss RL and the output of the power supply 192 will be described hereinafter with reference to FIG. 10. FIG. 10 includes an upper figure (a) schematically illustrating values of the return loss RLₘₑₐₛ acquired over time and a lower figure (b) schematically illustrating values of the output of the power supply 192 over time. It is assumed that the output switch 250 is ON at time t0. As depicted in the lower figure (b) of FIG. 10, the output level is set to the first output level by the processing of step S106 as described hereinbefore. At this time, the distal-end electrode 224 and the living tissue 900 are sufficiently spaced apart from each other. Therefore, the return loss RLₘₑₐₛ acquired in step S109 is of a large value. This value is stored as the maximum value RLₘₐₓ of the return loss RL.

After time t1, because the distal-end electrode 224 and the living tissue 900 gradually come closer to each other and an electric discharge is occurring therebetween, the return loss RLₘₑₐₛ gradually decreases. The return loss RLₘₑₐₛ at time t2, for example, is smaller than the maximum value RLₘₐₓ of the return loss RL. It is assumed that the difference between the return loss RLₘₑₐₛ and the maximum value RLₘₐₓ is equal to the second threshold value at time t3. At time t4 subsequent to time t3, the output level is changed to the second output level by the processing of step S114, as depicted in the lower figure (b) of FIG. 10. The second output level is depicted as zero.

The sensitivity with which to shift to the blanking period can be adjusted depending on how the second threshold value is set. Specifically, the sensitivity increases by reducing the second threshold value and decreases by increasing the second threshold value. The second threshold value can be set to an appropriate value. After time t4, as the distal-end electrode 224 and the living tissue 900 become much closer to each other, the return loss RLₘₑₐₛ further decreases. The blanking period in which the output level is the second output level is determined by the processing of steps S115 and S116. It is assumed that the blanking period elapses at time t5. At time t5, the output level is changed to the first output level by the processing of step S106. At time t6, when the distal-end electrode 224 and the living tissue 900 contact each other, the output level should desirably be the first output level. This is because as a treatment for incising the living tissue or stopping bleeding from the living tissue starts at time t6, the output level needs to be the output level desired by the user when the distal-end electrode 224 and the living tissue 900 contact each other at the latest.

The period from time t0 to time t1 is the period in which no incision is made in the living tissue, the period after time t6 is the period in which a treatment for incising the living tissue or stopping bleeding from the living tissue is performed, and the period from time t1 to time t6 is the transition period until the distal-end electrode 224 comes into contact with the living tissue 900. It is understood that sometime during the transition period, the output value may instantaneously deviate largely from a target value due, for example, to an unintended large electric discharge occurring between the distal-end electrode 224 and the living tissue 900. According to the present embodiment, as described hereinbefore, the start of an incision is predicted based on the acquisition of the return loss RL, and the output is temporarily reduced at a certain time in the transition period immediately before the incision. The temporary reduction in the output is effective to prevent the output value from instantaneously deviating largely from the target value. Modifications of the present embodiment will be set forth hereinafter.

Output Levels

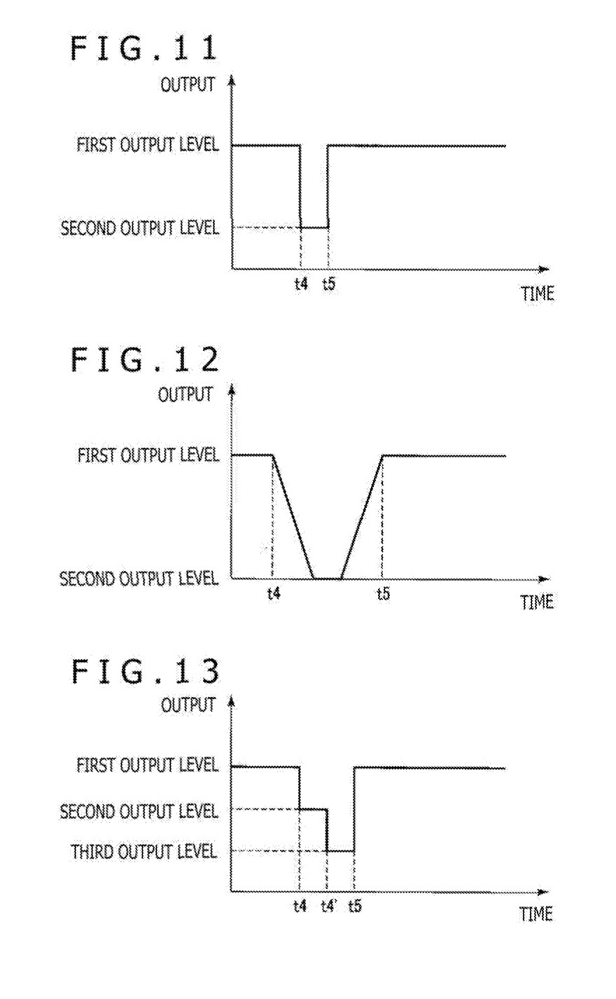

A modification relating to the output in the blanking period from time t4 to time t5 will be illustrated hereinafter. According to the embodiment described hereinbefore, the second output level is of an output value of zero, i.e., the output of the power supply 192 is stopped, in the blanking period. However, the second output level in the blanking period is not limited to such a value, but may be of a value lower than the first output level before and after the blanking period, i.e., a value not making the output deviating largely from the target value. For example, as depicted in FIG. 11, the second output level may be of a value lower than the first output level and higher than zero. In the blanking period, the output may thus be produced at the second output level, which may include zero, that is lower than the first output level.

The power supply device 100 may gradually change its output level from the first output level to the second output level as depicted in FIG. 12, rather than abruptly changing its output level from the first output level to the second output level in the blanking period according to the embodiment described hereinbefore. The power supply device 100 may also gradually change its output level from the second output level to the first output level. Generally, since the output level of an apparatus such as the treatment system 1 is large, it may produce electric noise when it abruptly changes its output level. The noise can thus be reduced by gradually changing the output level.

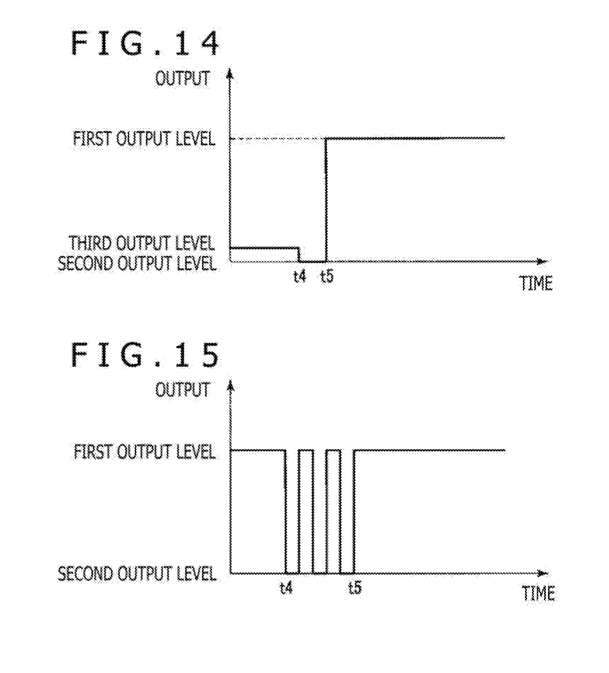

In the embodiment described hereinbefore, the output level switches between the first output level and the second output level in the blanking period. However, the output level is not limited to such output level switching. Rather, as depicted in FIG. 13, for example, the blanking period may be divided into a plurality of stages. Specifically, when a certain condition is met, the power supply device 100 changes its output level from a first output level to a second output level. Then, when another condition is met, the power supply device 100 changes the output level from the second output level to a third output level. When still another condition is met, the power supply device 100 changes the output level from the third output level to the first output level. The power supply device 100 may change the output level in several stages equal to or more than three stages. The power supply device 100 may change the output level gradually or may change the output level in other patterns. Moreover, as depicted in FIG. 14, the power supply device 100 may set its output level to a third output level that is low but enough to acquire the return loss RL prior to the blanking period. After the elapse of the blanking period, the power supply device 100 may set its output level to the first output level that is set by the user.

Furthermore, as depicted in FIG. 15, the power supply device 100 may change its output level alternately between the first output level and the second output level lower than the first output level, several times in the blanking period. In this case, the power supply device 100 may not repeat the second output level and the first output level for the purpose of reducing noise as described hereinbefore, but may repeat the second output level and the third output level lower than the first output level. The output value is prevented from instantaneously deviating largely from the target value by thus changing the output level at short intervals. In addition, even if the distal-end electrode 224 and the living tissue 900 come into contact with each other, entering an incision period, even before the blanking period ends, a certain capability for treating, such as incising or coagulating, the living tissue is maintained in the blanking period. Furthermore, the output level may be changed according to various patterns provided by combining the changing patterns of the output level described hereinbefore with reference to FIGS. 11 through 15.

Setting of Blanking Period

The blanking period is not limited to the preset time according to the embodiment described hereinbefore. The power supply device 100 may be arranged to change its output level to the first output level when the return loss RLₘₑₐₛ becomes smaller than a predetermined value, for example. Rather than using the relative value of the difference RLₘₐₓ−RLₘₑₐₛ in step S112, the return loss RLₘₑₐₛ which is of an absolute value, and a threshold may be compared with each other for determination. With such an arrangement, regardless of the speed at which the user moves the distal-end electrode 224, the output level is lowered to the second output level while the living tissue 900 and the distal-end electrode 224 are spaced from each other by a distance in a predetermined range.

Determination of Start of Blanking Period

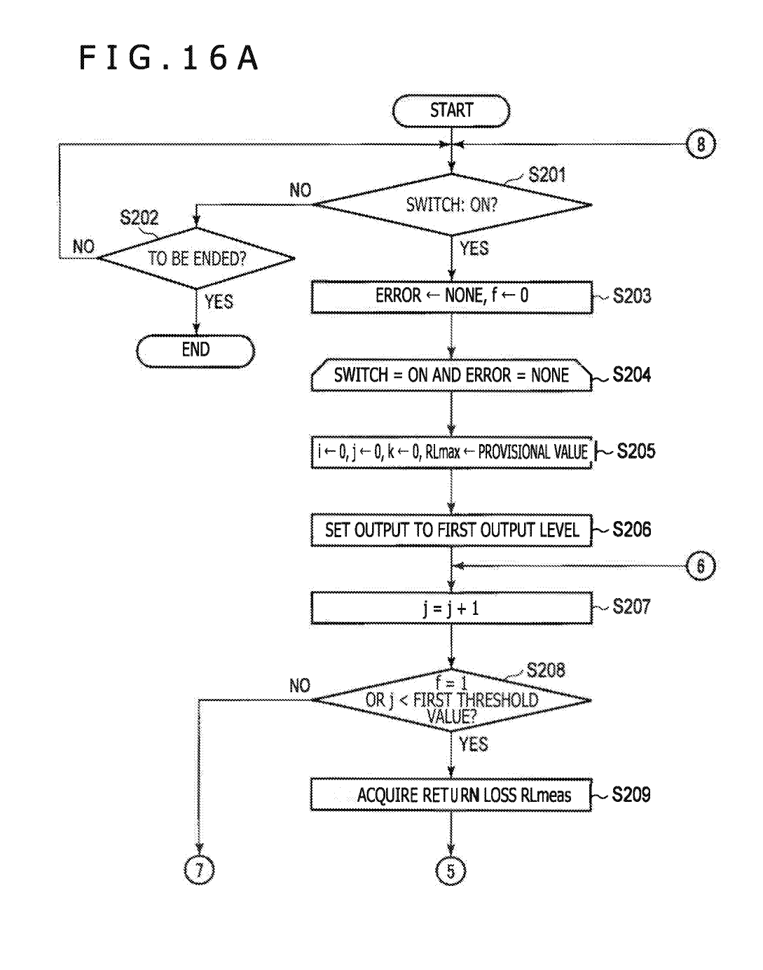

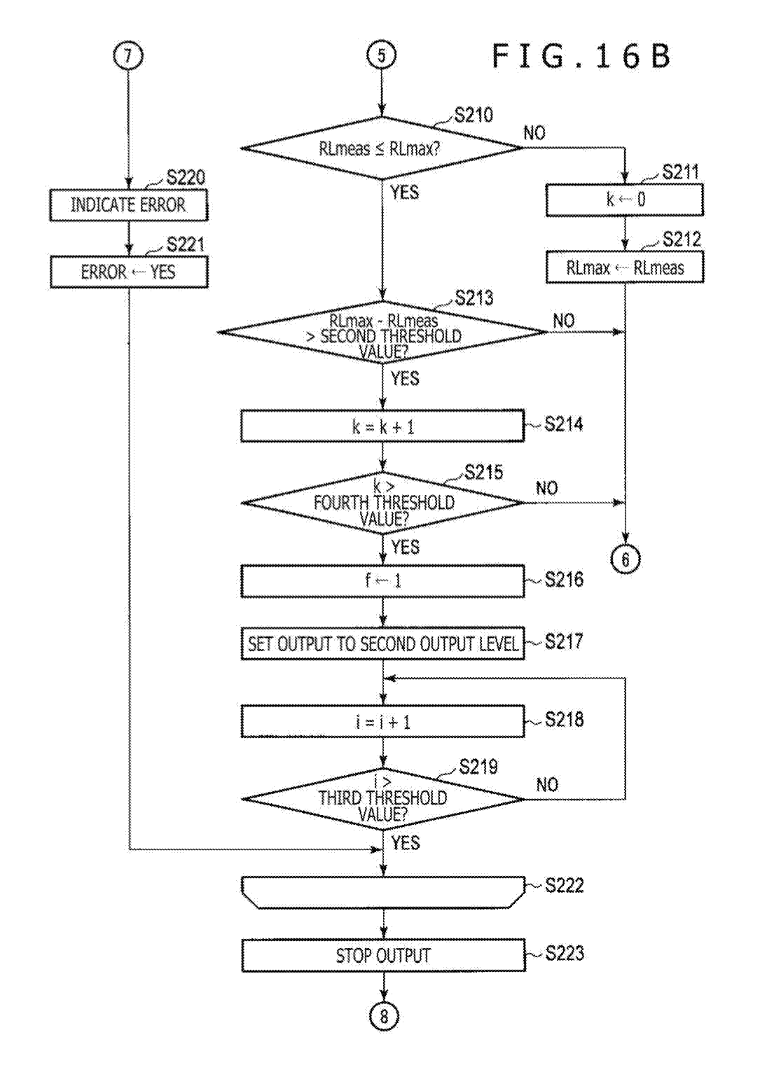

In the embodiment described hereinbefore, the power supply device 100 enters the blanking period if the difference RLₘₐₓ−RLₘₑₐₛ, obtained by subtracting the return loss RLₘₑₐₛ from the maximum value RLₘₐₓ of the return loss RL, is larger than the predetermined second threshold. However, if the power supply device 100 enters the blanking period when the condition is met only once, a determination error may possibly occur due to noise or the like. Therefore, the power supply device 100 may be arranged to enter the blanking period when the condition is met a certain number of times. A processing sequence for operating the power supply device 100 thus arranged will be described hereinafter with reference to a flowchart depicted in FIGS. 16A and 16B.

The operation of steps S201 through S204 is the same as the processing of steps S201 through S204 according to the embodiment described hereinbefore. Briefly stated, the output controller 164 determines in step S201 whether or not the output switch 250 is ON. If not ON, control goes to step S202. In step S202, the output controller 164 determines whether or not the present processing sequence is to be ended. If the present processing sequence is to be ended, then the present processing sequence is ended. If the present processing sequence is not to be ended, then control goes back to step S201. If it is determined in step S201 that the output switch 250 is ON, then control goes to step S203.

In step S203, the output controller 164 sets information relating to an error or no error to “NONE”, and sets a determination flag f to 0. The processing from step S204 to step S222 is an iteration that is carried out under the condition that the output switch 250 is ON and there is no error. If the output switch 250 is OFF or there is an error, then the iteration is bypassed, and control goes to step S223.

In step S205, the output controller 164 initializes variables stored in the memory 196. Specifically, the output controller 164 sets the first counter i for acquiring a blanking period, the second counter j for measuring a run-time error, and in addition a third counter k for avoiding a determination error, to 0. The output controller 164 also sets the maximum value RLₘₐₓ of the return loss RL to a provisional value.

The operation of steps S206 through S210 is the same as the processing of steps S106 through S110 according to the embodiment described hereinbefore. Briefly stated, the output controller 164 sets in step S206 the output level of the power supply 192 to a first output level. In step S207, the output controller 164 increments the value of the second counter j. In step S208, the output controller 164 determines whether or not the determination flag f is 1 or the second counter j is smaller than a predetermined first threshold value. If the determination flag f is 1 or the second counter j is smaller than the first threshold value, then control goes to step S209. In step S209, the output controller 164 acquires the return loss RLₘₑₐₛ in step S209.

In step S210, the output controller 164 determines whether or not the return loss RLₘₑₐₛ is equal to or smaller than the present maximum value RLₘₐₓ. If the return loss RLₘₑₐₛ is not equal to or smaller than the maximum value RLₘₐₓ, then control goes to step S211. In step S211, the output controller 164 resets the value of the third counter k to 0. In step S212, the output controller 164 sets the maximum value RLₘₐₓ to the return loss RLₘₑₐₛ. Thereafter, control goes back to step S207. If it is determined in step S210 that the return loss RLₘₑₐₛ is equal to or smaller than the maximum value RLₘₐₓ, then control goes to step S213. In step S213, the output controller 164 determines whether or not the difference RLₘₐₓ−RLₘₑₐₛ, obtained by subtracting the return loss Rlₘₑₐₛ from the maximum value RLₘₐₓ of the return loss RL, is larger than a predetermined second threshold value. If the difference RLₘₐₓ−RLₘₑₐₛ is not larger than the second threshold value, then control returns to step S207. If the difference RLₘₐₓ−RLₘₑₐₛ is larger than the second threshold value, then control goes to step S214.

In step S214, the output controller 164 increments the value of the third counter k stored in the memory 196. In step S215, the output controller 164 determines whether or not the third counter k is larger than a predetermined fourth threshold value. If the third counter k is not larger than the fourth threshold value, then control goes back to step S207. If the third counter k is larger than the fourth threshold value, then control goes to step S216.

According to the present modification, control goes to step S216 for the first time if the number of times that the difference RLₘₐₓ−RLₘₑₐₛ, obtained by subtracting the return loss RLₘₑₐₛ from the maximum value RLₘₐₓ of the return loss RL, is determined as being larger than the second threshold value is larger than the fourth threshold value. Since control goes to steps S216 and S217 when the difference RLₘₐₓ−RLₘₑₐₛ is repeatedly determined as being larger than the second threshold value, a determination error due to noise or the like is prevented.

If the determination flag f is not 1 and the second counter j is equal to or larger than the first threshold value in step S208, then control goes to step S220. In step S220, the output controller 164 indicates an error representing that the distal-end electrode 224 has not been in contact with the living tissue 900 for a certain period though the output switch 250 is ON. In step S221, the output controller 164 sets the information relating to an error or no error to “YES”. Thereafter, control goes to step S222. Since the information relating to an error or no error has been set to “YES”, control goes to step S223. In step S223, the output controller 164 stops the output of the power supply 192. Thereafter, control goes back to step S201.

The processing of steps S216 through S223 is the same as the processing of steps S113 through S120 according to the embodiment described hereinbefore. Briefly stated, the output controller 164 sets the determination flag f to 1 in step S216. In step S217, the output controller 164 sets the output level of the power supply 192 to a second output level. In step S218, the output controller 164 increments the first counter i. In step S219, the output controller 164 determines whether or not the first counter i is larger than a predetermined third threshold value. If the first counter i is not larger than the third threshold value, then control goes back to step S218. In other words, the processing of steps S218 and S219 is repeated until the first counter i exceeds the third threshold value. If it is determined in step S219 that the first counter i is larger than the third threshold, then control goes to step S222. In other words, the processing from step S204 is repeated when the switch is ON and there is no error.

According to the present modification, it is possible to adjust the sensitivity with which to switch between output levels by introducing the fourth threshold value. Though whether or not the difference RLₘₐₓ−RLₘₑₐₛ, obtained by subtracting the return loss RLₘₑₐₛ from the maximum value RLₘₐₓ of the return loss RL, is larger than the predetermined second threshold value is used as a determination criterion herein, the present modification is not limited to such a determination criterion. Instead, whether or not the absolute value of the return loss RLₘₑₐₛ meets a predetermined condition may be used as a determination criterion.

According to the second embodiment, the treatment system 1 operates to stop the output temporarily when the distance between the distal-end electrode 224 and the living tissue to be treated reaches a predetermined distance while the distal-end electrode 224 is moving away from the living tissue.

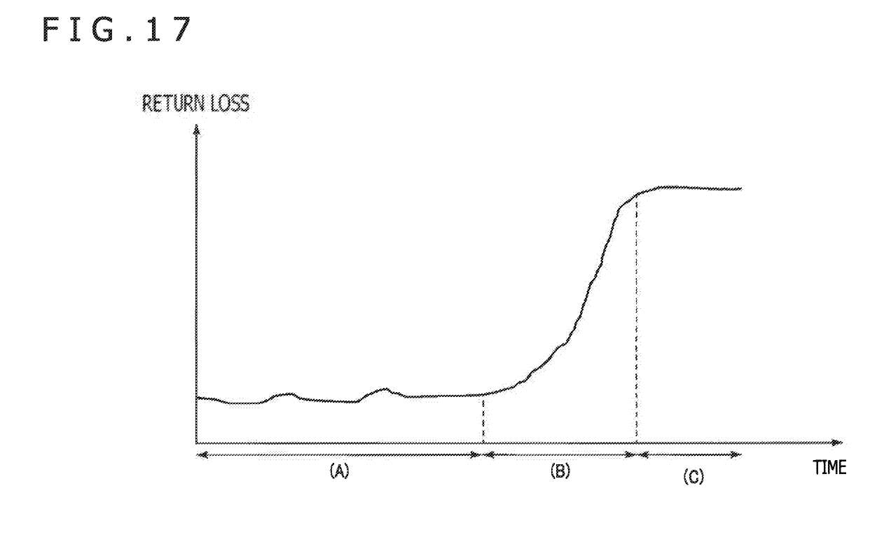

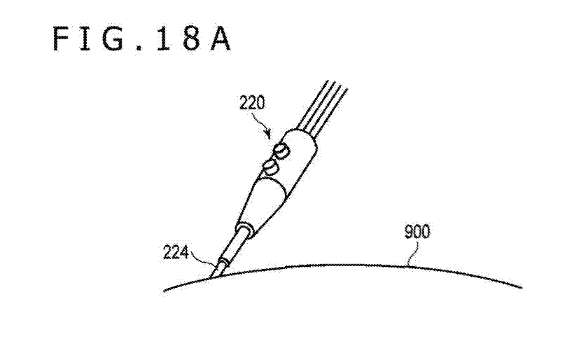

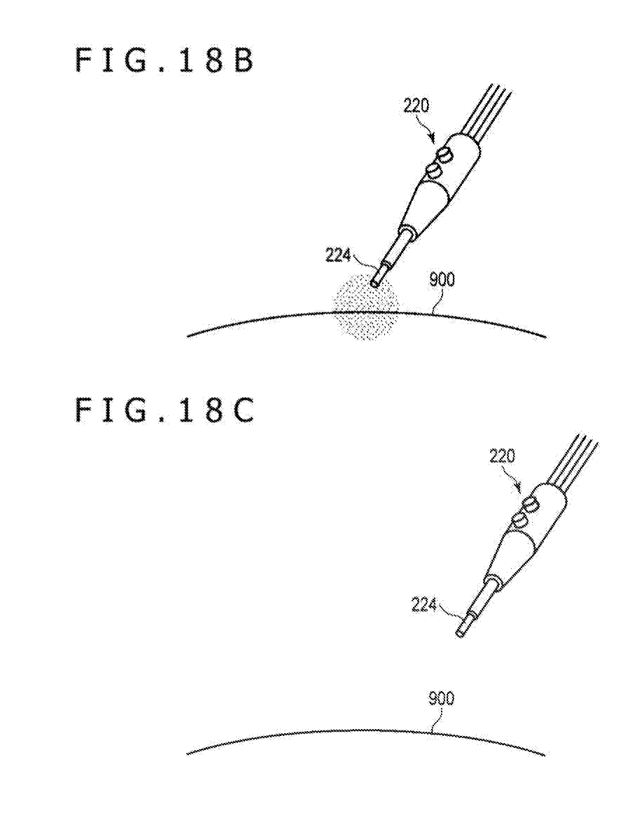

An outline of operation of the power supply device 100 according to the present embodiment will be described hereinafter with reference to FIGS. 17, 18A, 18B, and 18C. FIG. 17 has a horizontal axis representing time and a vertical axis representing the return loss RL calculated by the return loss acquirer 162. FIG. 17 illustrates the relation between time and the return loss RL as the distal-end electrode 224 moves gradually away from the living tissue to be treated from the state of being in contact therewith while a high-frequency voltage is being applied between the distal-end electrode 224 and the counter electrode plate 240. During a period indicated by (A) in FIG. 17, the distal-end electrode 224 is in contact with the living tissue 900, as depicted in FIG. 18A. At this time, the return loss RL is of a relatively small value. During a period indicated by (B) in FIG. 17, the distal-end electrode 224 is moving away from the living tissue 900, bringing about an electric discharge between the distal-end electrode 224 and the living tissue 900. The return loss RL acquired during this period is higher than the return loss RL that is acquired in the period indicated by (A) in FIG. 17. FIG. 18B schematically illustrates the situation at a time included in the period indicated by (B) in FIG. 17, where an electric discharge is recognized in a region, depicted stipulated in FIG. 18B, between the distal-end electrode 224 and the living tissue 900. During a period indicated by (C) in FIG. 17, there is a sufficient distance between the distal-end electrode 224 and the living tissue 900, as depicted in FIG. 18C. During this period, the return loss RL is of a large value.

It is understood that during a part of the period indicated by (B) in FIG. 17, i.e., during a partial period while an electric discharge is occurring between the distal-end electrode 224 and the living tissue 900 (a period in which a large electric discharge occurs while the distance between the distal-end electrode 224 and the living tissue 900 is in a particular range), the control process tends to become unstable due, for example, to an unintended excessive output current that flows. According to the present embodiment, the output is stopped during a part of the period indicated by (B) in FIG. 17.

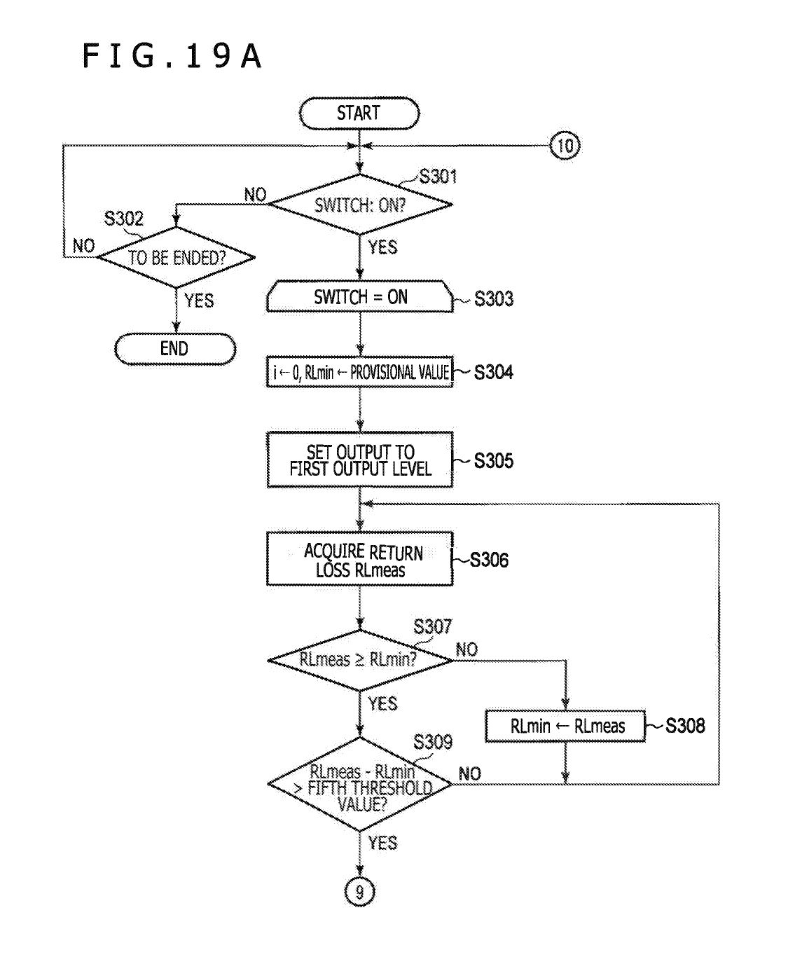

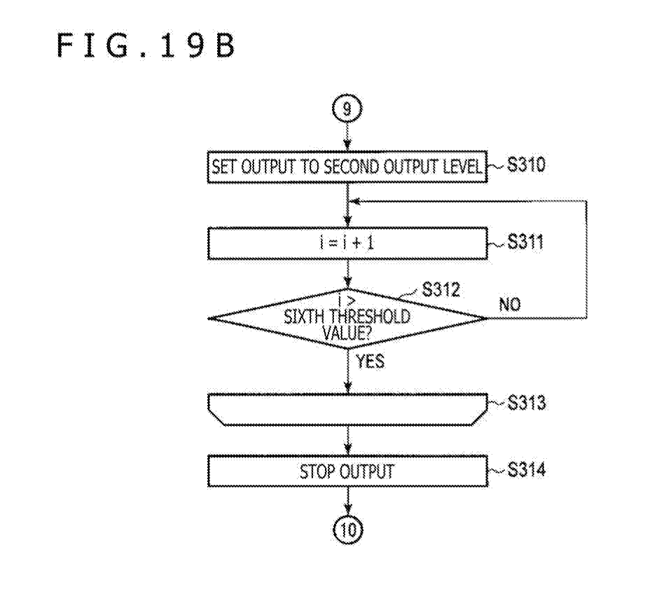

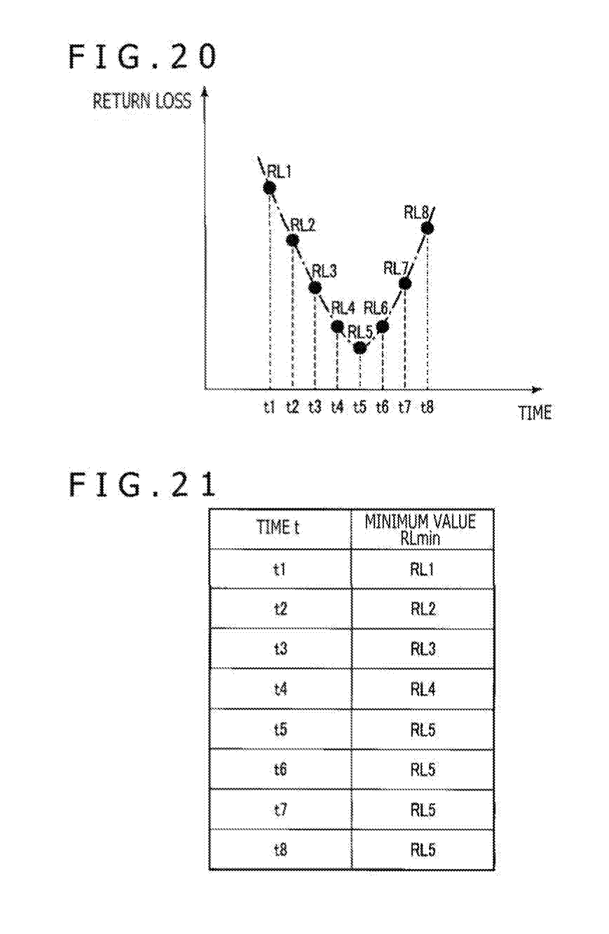

Operation of the power supply device 100 according to the present embodiment will be described hereinafter with reference to a flowchart depicted in FIGS. 19A and 19B. The present processing sequence is carried out when the power supply device 100 has its main power supply turned on.

In step S301, the output controller 164 determines whether or not the output switch 250, which represents the foot switch assembly 260 or the hand switch assembly 226 for giving a command to turn on or off the output, is ON. If the output switch 250 is not ON, then control goes to step S302. In step S302, the output controller 164 determines whether or not the present processing sequence is to be ended, e.g., whether or not the main power supply is turned off. If the present processing sequence is to be ended, then the present processing sequence is ended. If the present processing sequence is not to be ended, then control goes back to step S301. In other words, as long as the output switch 250 is OFF, control waits by repeating steps S301 and S302. If it is determined in step S301 that the output switch 250 is ON, then control goes to step S303.

The processing from step S303 to step S313 is an iteration that is carried out under the condition that the output switch 250 is ON. If the output switch 250 is OFF, then the iteration is bypassed, and control goes to step S314.

In step S304, the output controller 164 initializes variables stored in the memory 196. Specifically, the output controller 164 sets a first counter i for measuring a blanking period, to be described hereinafter, to 0. The output controller 164 also sets a minimum value RLₘᵢₙ of the return loss RL, to be described hereinafter, to a provisional value. It is desirable that the provisional value should be of a value sufficiently larger than a value expected to be the minimum value RLₘᵢₙ.

In step S305, the output controller 164 sets the output level of the power supply 192 to a first output level. The first output level is an output level, set by the user, for example, that is required for a treatment. The output level may be controlled by a voltage control process, a current control process, or other processes. Since the output level is set to the first output level, the user can treat the living tissue by bringing the distal-end electrode 224 into contact with the living tissue 900.

In step S306, the output controller 164 acquires a measured return loss RLₘₑₐₛ based on a voltage value acquired by the detection circuit 110.

In step S307, the output controller 164 determines whether or not the return loss RLₘₑₐₛ is equal to or larger than the minimum value RLₘᵢₙ of the return loss RL stored in the memory 196 at the time. If the return loss RLₘₑₐₛ is not equal to or larger than the minimum value RLₘᵢₙ, then control goes to step S308.

In step S308, the output controller 164 sets the minimum value RLₘᵢₙ to the value of the return loss RLₘₑₐₛ. In other words, the minimum value RLₘᵢₙ is updated. Since the return loss RLₘₑₐₛ can be monotonously increased or reduced, rather than being monotonously reduced, the minimum value RLₘᵢₙ of the return loss RL is updated as by the processing of step S308. For example, as depicted in FIG. 20, it is assumed that as time t elapses to t1, t2, t3, t4, and t5, the return loss RL gradually decreases to RL1, RL2, RL3, RL4, and RL5. At this time, as depicted in FIG. 21, the minimum value RLₘᵢₙ of the return loss RL gradually decreases to RL1, RL2, RL3, RL4, and RL5. After the processing of step S308, control returns to step S306.

If it is determined in step S307 that the return loss RLₘₑₐₛ is equal to or larger than the minimum value RLₘᵢₙ, then control goes to step S309. For example, as depicted in FIG. 20, it is assumed that as time t elapses to t5, t6, t7, and t8, the return loss RLₘₑₐₛ gradually increases to RL5, RL6, RL7, and RL8. At this time, the minimum value RLₘᵢₙ of the return loss RL remains to be RL5, and is not updated.

In step S309, the output controller 164 determines whether or not the difference RLₘₑₐₛ−RLₘᵢₙ, obtained by subtracting the minimum value RLₘᵢₙ of the return loss RL from the return loss RLₘₑₐₛ, is larger than a predetermined fifth threshold value. If the difference RLₘₑₐₛ−RLₘᵢₙ is not larger than the fifth threshold value, then control returns to step S306.

When the user moves the distal-end electrode 224 gradually away from the living tissue 900, for example, the minimum value RLₘᵢₙ is not changed, and the difference RLₘₑₐₛ−RLₘᵢₙ becomes gradually larger.

If it is determined in step S309 that the difference RLₘₑₐₛ−RLₘᵢₙ is larger than the fifth threshold value, then control goes to step S310. The condition that the difference RLₘₑₐₛ−RLₘᵢₙ is larger than the fifth threshold value corresponds to a switching condition for shifting to a restrained state where the output is reduced. In step S310, the output controller 164 sets the output level of the power supply 192 to a second output level. Though the second output level will be described as zero, it may not be zero. If the output is zero, the output controller 164 stops the output of the power supply 192.

In step S311, the output controller 164 increments a first counter i stored in the memory 196.

In step S312, the output controller 164 determines whether or not the first counter i is equal to or larger than a sixth threshold value. If the first counter i is not larger than the six threshold value, then control goes back to step S311. In other words, the processing of steps S311 and S312 is repeated until the first counter i exceeds the sixth threshold value. Stated otherwise, control waits for a predetermined period. The period counted by the first counter i, i.e., the period during which the output is stopped, will be referred to as a blanking period. The blanking period is ten milliseconds, for example. The state in which the output level is the second output level during the blanking period corresponds to the restrained state when the return loss satisfies the given switching condition.

If it is determined in step S312 that the first counter i is larger than the sixth threshold value, control goes to step S313. If the switch is ON and there is no error, the processing from step S303 is repeated.

After the switching condition has been met and the output level has been set to the second output level in the period indicated by (B) in FIG. 17, the processing that begins with step S303 is carried out again. In step S305, the output level of the power supply 192 is set again to the first output level. The minimum value RLₘᵢₙ of the return loss RL is set again to the provisional value in step S304. Since the acquired return loss RLₘₑₐₛ increases, the difference RLₘₑₐₛ−RLₘᵢₙ between the return loss RLₘₑₐₛ and the minimum value RLₘᵢₙ may become larger than the fifth threshold value depending on how the fifth threshold value is set. During the period indicated by (C) in FIG. 17, since there is a sufficient distance between the living tissue 900 and the distal-end electrode 224, as depicted in FIG. 18C, and the distal-end electrode 224 does not incise the living tissue 900 or does not stop bleeding from the living tissue 900, the second output level may be zero.

If the output switch 250 is OFF, then control goes to step S314. In step S314, the output controller 164 stops the output of the power supply 192. Thereafter, control returns to step S301.

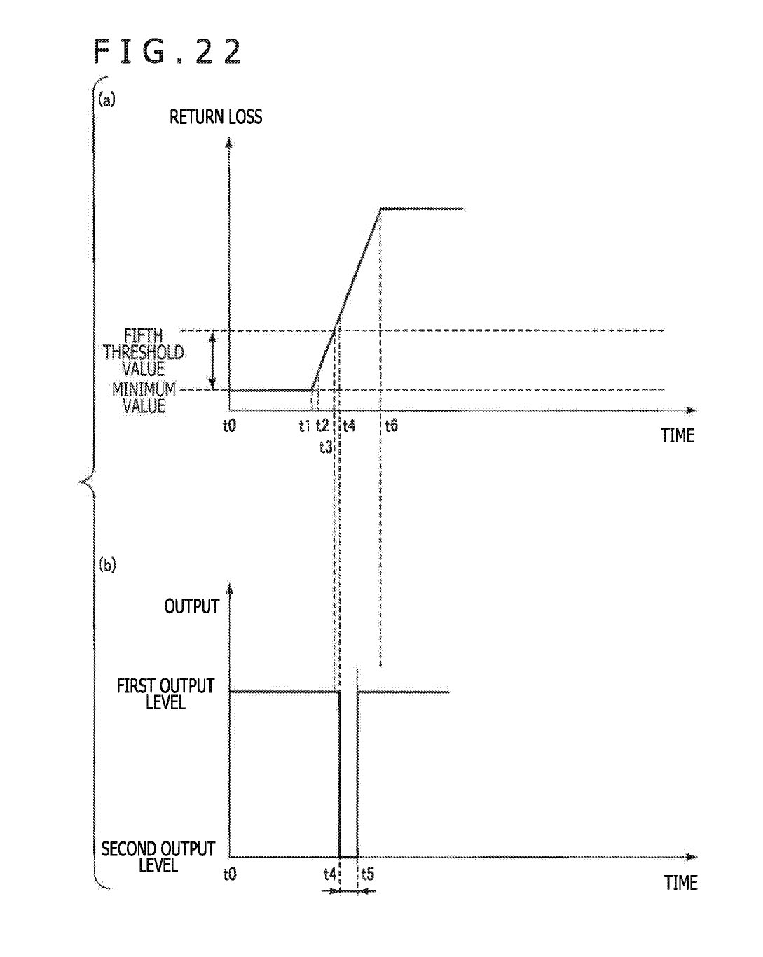

Time-dependent changes of the acquired return loss and the output will be described hereinafter with reference to FIG. 22. FIG. 22 includes an upper figure (a) schematically illustrating values of the return loss RLₘₑₐₛ acquired over time and a lower figure (b) schematically illustrating values of the output of the power supply 192 over time.

It is assumed that the output switch 250 is ON at time t0. As depicted in the lower figure (b) of FIG. 22, the output level is set to the first output level by the processing of step S305 as described hereinbefore. At this time, the distal-end electrode 224 and the living tissue 900 are in contact with each other. Therefore, the return loss RLₘₑₐₛ acquired in step S306 is of a small value. This value is stored as the minimum value RLₘᵢₙ of the return loss RL.

After time t1, the distal-end electrode 224 and the living tissue 900 gradually move away from each other. At this time, an electric discharge occurs between the distal-end electrode 224 and the living tissue 900. The return loss RLₘₑₐₛ gradually increases. The return loss RLₘₑₐₛ at time t2, for example, is larger than the minimum value RLₘᵢₙ of the return loss RL.

It is assumed that the difference between the return loss RLₘₑₐₛ and the minimum value RLₘᵢₙ is equal to the fifth threshold value at time t3. At time t4 subsequent to time t3, the output level is changed to the second output level by the processing of step S310, as depicted in the lower figure (b) of FIG. 22. The second output level is depicted as zero. The output level may be changed to the second output level at time t3. The sensitivity with which to shift to the blanking period can be adjusted depending on how the fifth threshold value is set. Specifically, the sensitivity increases by decreasing the fifth threshold value and decreases by increasing the fifth threshold value. The fifth threshold value can be set to an appropriate value.

After time t4, as the distal-end electrode 224 and the living tissue 900 move further away from each other, the return loss RLₘₑₐₛ further increases. The blanking period in which the output level is the second output level is determined by the processing of steps S311 and S312. It is assumed that the blanking period elapses at time t5. At time t5, the output level is changed to the first output level by the processing of step S305. As time goes on and after time t6, the distal-end electrode 224 and the living tissue 900 are sufficiently spaced from each other, and the return loss RLₘₑₐₛ is of a sufficiently large value.

The period from time t0 to time t1 is the period in which a treatment for incising the living tissue or stopping bleeding from the living tissue is performed, the period after time t6 is the period in which no treatment is performed, and the period from time t1 to time t6 is the transition period in which the distal-end electrode 224 moves away from the living tissue 900. It is understood that sometime during the transition period, the output value of the power supply 192 may instantaneously deviate largely from a target value due, for example, to an unintended large electric discharge occurring between the distal-end electrode 224 and the living tissue 900. According to the present embodiment, as described hereinbefore, movement of the distal-end electrode 224 away from the living tissue 900 is detected based on the acquired return loss RL, and the output is temporarily reduced at a certain time in the transition period. The temporary reduction in the output is effective to prevent the output value from instantaneously deviating largely from the target value.

Modifications of the present embodiment will be set forth hereinafter.

Output Levels

A modification relating to the output in the blanking period from time t4 to time t5 will be illustrated hereinafter. According to the embodiment described hereinbefore, the second output level is of an output value of zero, i.e., the output of the power supply 192 is stopped, in the blanking period. However, the second output level in the blanking period is not limited to such a value, but may be appropriately changed as is the case with the first embodiment. For example, the second output level in the blanking period may be of a value lower than the first output level before and after the blanking period, i.e., a value not making the output deviating largely from the target value. For example, as depicted in FIG. 11, the second output level may be of a value lower than the first output level and higher than zero. In the blanking period, the output may thus be produced at the second output level, which may include zero, that is lower than the first output level.

The power supply device 100 may gradually change its output level from the first output level to the second output level as depicted in FIG. 12, rather than abruptly changing from the first output level to the second output level in the blanking period according to the embodiment described hereinbefore. The power supply device 100 may also gradually change the output from the second output level to the first output level. The noise can thus be reduced by gradually changing the output level.

As depicted in FIG. 13, for example, the blanking period may be divided into a plurality of stages. Specifically, when a certain condition is met, the power supply device 100 changes its output level from a first output level to a second output level. Then, when another condition is met, the power supply device 100 changes the output level from the second output level to a third output level. When still another condition is met, the power supply device 100 changes the output level from the third output level to the first output level. The power supply device 100 may change the output level in several stages equal to or more than three stages. The power supply device 100 may change the output level gradually or may change the output level in other patterns.

Furthermore, as depicted in FIG. 15, the power supply device 100 may change the output level alternately between the first output level and the second output level lower than the first output level, several times in the blanking period. In this case, the power supply device 100 may not repeat the second output level and the first output level for the purpose of reducing noise as described hereinbefore, but may repeat the second output level and the third output level lower than the first output level. The output value is prevented from instantaneously deviating largely from the target value by thus changing the output level at short intervals.

Furthermore, the output level may be changed according to various patterns provided by combining the changing patterns of the output level described hereinbefore with reference to FIGS. 11, 12, 13, and 15.

Setting of Blanking Period

The blanking period is not limited to the preset time according to the embodiment described hereinbefore. The power supply device 100 may be arranged to change its output level to the first output level when the return loss RLₘₑₐₛ becomes larger than a predetermined value, for example. Rather than using the relative value of the difference RLₘₑₐₛ−RLₘᵢₙ in step S309, the absolute value of only the return loss RLₘₑₐₛ and a threshold value may be compared with each other for determination. With such an arrangement, regardless of the speed at which the user moves the distal-end electrode 224, the output level is lowered to the second output level while the living tissue 900 and the distal-end electrode 224 are spaced from each other by a distance in a predetermined range.

Determination of Start of Blanking Period

In the embodiment described hereinbefore, the power supply device 100 enters the blanking period if the difference RLₘₑₐₛ−RLₘᵢₙ, obtained by subtracting the minimum value RLₘᵢₙ of the return loss RL from the return loss RLₘₑₐₛ, is larger than the predetermined fifth threshold. However, such a condition is not restrictive. An average value of the return loss RL that is acquired when the distal-end electrode 224 is in contact with the living tissue 900 in the period indicated by (A) in FIG. 17 is represented by RL_{average} and the power supply device 100 may enter the blanking period if the difference RLₘₑₐₛ−RL_{average}, obtained by subtracting the average value RL_{average} of the return loss RL from the return loss RLₘₑₐₛ, is larger than the predetermined fifth threshold value. Providing the power supply device 100 enters the blanking period when a value larger than the return loss RL acquired when the distal-end electrode 224 is in contact with the living tissue 900 is acquired, the condition for entering the blanking period can flexibly be changed even if the condition of the living tissue has changed.

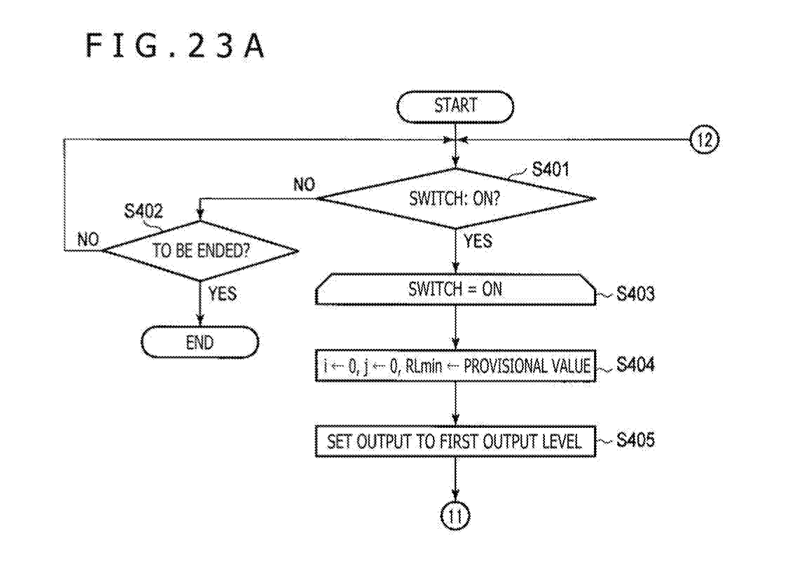

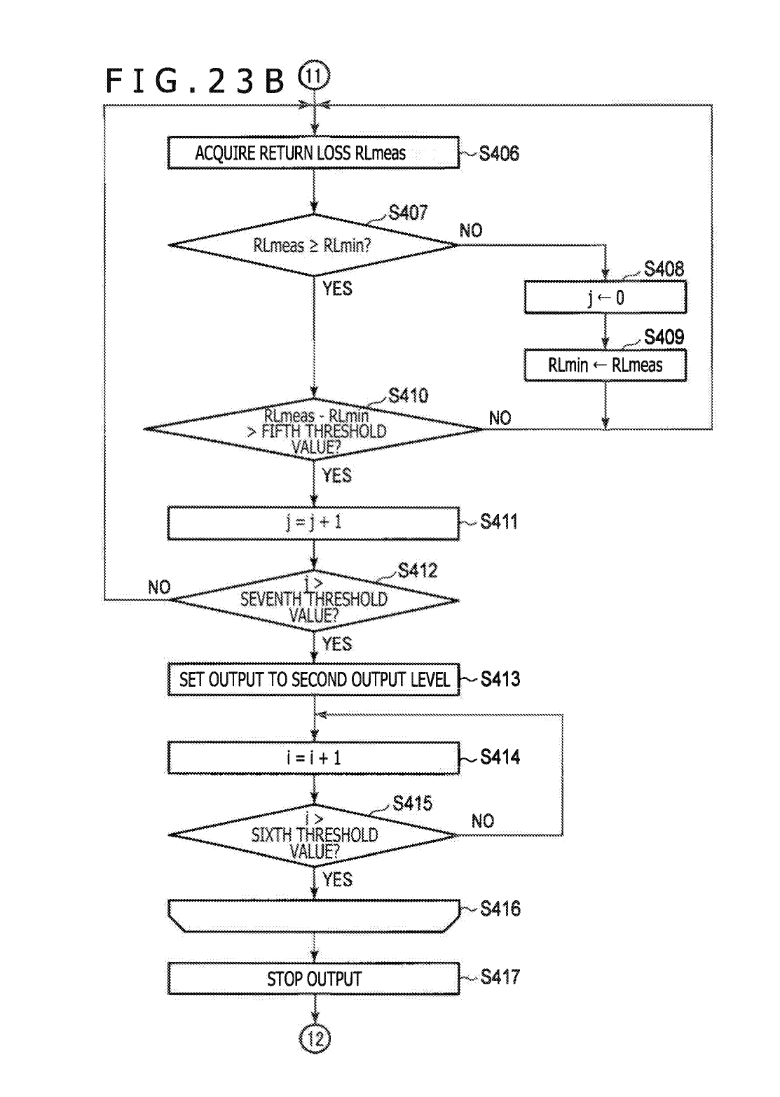

In the example described hereinbefore, the power supply device 100 enters the blanking period when the absolute value of the difference RLₘₑₐₛ−RLₘᵢₙ is larger than the predetermined fifth threshold value. However, if the power supply device 100 enters the blanking period when the condition is met only once, a determination error may possibly occur due to noise or the like. Therefore, the power supply device 100 may be arranged to enter the blanking period when the condition is met a certain number of times. A processing sequence for operating the power supply device 100 thus arranged will be described hereinafter with reference to a flowchart depicted in FIGS. 23A and 23B.

The operation of steps S401 through S403 is the same as the processing of steps S301 through S303 according to the embodiment described hereinbefore. Briefly stated, the output controller 164 determines in step S401 whether or not the output switch 250 is ON. If not ON, control goes to step S402. In step S402, the output controller 164 determines whether or not the present processing sequence is to be ended. If the present processing sequence is to be ended, then the present processing sequence is ended. If the present processing sequence is not to be ended, then control goes back to step S401. If it is determined in step S401 that the output switch 250 is ON, then control goes to step S403.

The processing from step S403 to step S416 is an iteration that is carried out under the condition that the output switch 250 is ON. If the output switch 250 is OFF, then the iteration is bypassed, and control goes to step S417.

In step S404, the output controller 164 initializes variables stored in the memory 196. Specifically, the output controller 164 sets a first counter i for measuring a blanking period to 0, and in addition also sets the value of a second counter j for avoiding a determination error due to noise or the like to 0. The output controller 164 also sets a minimum value RLₘᵢₙ of the return loss RL to a provisional value.

The operation of steps S405 through S407 is the same as the processing of steps S305 through S307 according to the embodiment described hereinbefore. Briefly stated, the output controller 164 sets in step S405 the output level of the power supply 192 to a first output level. In step S406, the output controller 164 acquires the return loss RLₘₑₐₛ.

In step S407, the output controller 164 determines whether or not the return loss RLₘₑₐₛ is equal to or larger than the present minimum value RLₘᵢₙ. If the return loss RLₘₑₐₛ is not equal to or larger than the minimum value RLₘᵢₙ, then control goes to step S408.

In step S408, the output controller 164 resets the value of the second counter j to 0. In step S409, the output controller 164 sets the minimum value RLₘᵢₙ to the return loss RLₘₑₐₛ. Thereafter, control goes back to step S406.

If it is determined in step S407 that the return loss RLₘₑₐₛ is equal to or larger than the minimum value RLₘᵢₙ, then control goes to step S410. In step S410, the output controller 164 determines whether or not the difference RLₘₑₐₛ−RLₘᵢₙ, obtained by subtracting the minimum value RLₘᵢₙ of the return loss RL from the return loss RLₘₑₐₛ is larger than a predetermined fifth threshold value. If the difference RLₘₑₐₛ−RLₘᵢₙ is not larger than the fifth threshold value, then control returns to step S406. If the difference RLₘₑₐₛ−RLₘᵢₙ is larger than the fifth threshold value, then control goes to step S411.

In step S411, the output controller 164 increments the value of the second counter j stored in the memory 196.

In step S412, the output controller 164 determines whether or not the second counter j is larger than a predetermined seventh threshold value. If the second counter j is not larger than the seventh threshold value, then control goes back to step S406. If the second counter j is larger than the seventh threshold value, then control goes to step S413.

According to the present modification, control goes to step S413 for the first time if the number of times that the difference RLₘₑₐₛ−RLₘᵢₙ, obtained by subtracting the minimum value RLₘᵢₙ of the return loss RL from the return loss RLₘₑₐₛ is determined as being larger than the fifth threshold value is larger than the seventh threshold value. Since control goes to step S413 when the difference RLₘₑₐₛ RLₘᵢₙ is repeatedly determined as being larger than the fifth threshold value, the processing of an unintended output level change due to noise or the like is prevented.

The processing of steps S413 through S417 is the same as the processing of steps S310 through S314 according to the embodiment described hereinbefore. Briefly stated, the output controller 164 sets in step S413 the output level of the power supply 192 to a second output level. In step S414, the output controller 164 increments the first counter i. In step S415, the output controller 164 determines whether or not the first counter i is larger than a predetermined sixth threshold value. If the first counter i is not larger than the sixth threshold value, then control goes back to step S414. In other words, the processing of steps S414 and 415 is repeated until the first counter i exceeds the sixth threshold value. If the first counter i is determined as being larger than the sixth threshold value in step S415, control goes to step S416. In other words, the processing from step S403 is repeated when the switch is ON.

According to the present modification, it is possible to adjust the sensitivity with which to switch between output levels by introducing the seventh threshold value depicted in FIG. 23B. Though whether or not the difference RLₘₑₐₛ−RLₘᵢₙ, obtained by subtracting the minimum value RLₘᵢₙ of the return loss RL from the return loss RLₘₑₐₛ is larger than the predetermined fifth threshold value is used as a determination criterion herein, the present modification is not limited to such a determination criterion. Instead, whether or not the absolute value of the return loss RLₘₑₐₛ meets a predetermined condition may be used as a determination criterion.

According to the third embodiment, the treatment system 1 operates so as to cause the output controller 164 to reduce the output of the power supply 192 when the distance between the distal-end electrode 224 and the living tissue to be treated reaches a predetermined distance while the distal-end electrode 224 is moving closer to the living tissue and while the distal-end electrode 224 is moving away from the living tissue.

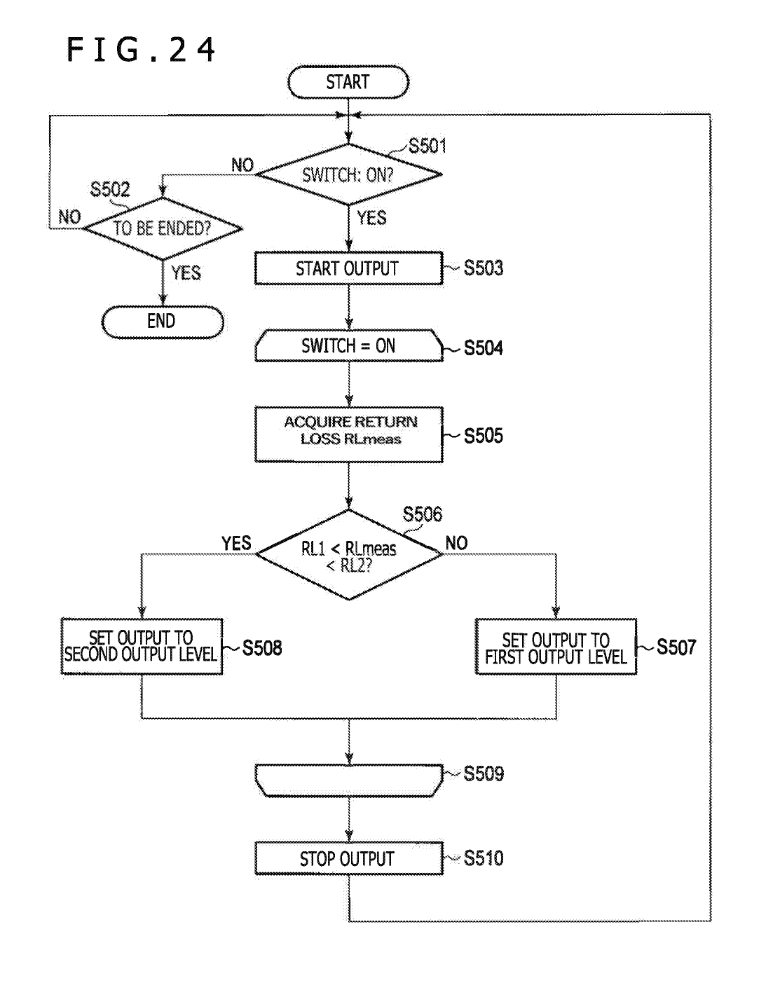

Operation of the power supply device 100 according to the present embodiment will be described hereinafter with reference to a flowchart depicted in FIG. 24. The present processing sequence is carried out when the power supply device 100 has its main power supply turned on.

In step S501, the output controller 164 determines whether or not the output switch 250, which represents the foot switch assembly 260 or the hand switch assembly 226 for giving a command to turn on or off the output, is ON. If the output switch 250 is not ON, then control goes to step S502. In step S502, the output controller 164 determines whether or not the present processing sequence is to be ended, e.g., whether or not the main power supply is turned off. If the present processing sequence is to be ended, then the present processing sequence is ended. If the present processing sequence is not to be ended, then control goes back to step S501. In other words, as long as the output switch 250 is OFF, control waits by repeating steps S501 and S502.

If it is determined in step S501 that the output switch 250 is ON, then control goes to step S503. In step S503, the output controller 164 controls the power supply 192 to start producing its output. The output is set to a first output level, to be described hereinafter, for example.

The processing from step S504 to step S509 is an iteration that is carried out under the condition that the output switch 250 is ON. If the output switch 250 is OFF, then the iteration is bypassed, and control goes to step S510. In step S505, the output controller 164 acquires a return loss RLₘₑₐₛ based on a voltage value acquired by the detection circuit 110.

In step S506, the output controller 164 determines whether or not the return loss RLₘₑₐₛ is larger than a predetermined first value RL1 and smaller than a predetermined second value RL2. The first value RL1 and the second value RL2 are a lower limit value and an upper limit value, respectively, that RL can take when an unintended large electric discharge can occur between the distal-end electrode 224 and the living tissue 900.

If RL1<RLₘₑₐₛ<RL2 is not met, then control goes to step S507. In step S507, the output controller 164 operates the power supply 192 at a first output level that is an output level for treating the living tissue 900. Thereafter, control goes to step S509. In other words, providing the output switch 250 is ON, the processing of steps S504 through S509 is repeated again.

If RL1<RLₘₑₐₛ<RL2 is met, then control goes to step S508. In step S508, the output controller 164 operates the power supply 192 at a second output level that is an output level in a restrained state lower than the first output level. Thereafter, control goes to step S509. In other words, providing the output switch 250 is ON, the processing of steps S504 through S509 is repeated again.

In other words, if there is a possibility that the output value may instantaneously deviate largely from a target value due, for example, to an unintended large electric discharge occurring between the distal-end electrode 224 and the living tissue 900 based on the return loss RLₘₑₐₛ, then the output level of the power supply 192 is reduced. Otherwise, the output level of the power supply 192 is set to the first output level that is an output level for treating the living tissue 900.

If the output switch 250 is OFF, then control goes to step S510. In step S510, the output controller 164 stops the output of the power supply 192, after which control goes back to step S501.

According to the present embodiment, the output is temporarily reduced when the distance between the distal-end electrode 224 and the living tissue 900 to be treated reaches a predetermined distance based on the acquired return loss RL. The temporary reduction in the output is effective to prevent the output value from instantaneously deviating largely from the target value.

According to the present embodiment, as with the first embodiment and the second embodiment, the output level of the power supply 192 may be appropriately changed as described hereinbefore with reference to FIGS. 11 through 15. The treatment system 1 may include various error detecting mechanisms as is the case with the first embodiment and the second embodiment.

High-Frequency Treatment Tools

In the embodiments described hereinbefore, for example the treatment tool 220 is illustrated as a monopolar high-frequency treatment tool. However, the treatment tool 220 may be a bipolar treatment tool. In this case, the two electrodes of the treatment tool correspond to the distal-end electrode 224 and the counter electrode plate 240, respectively.

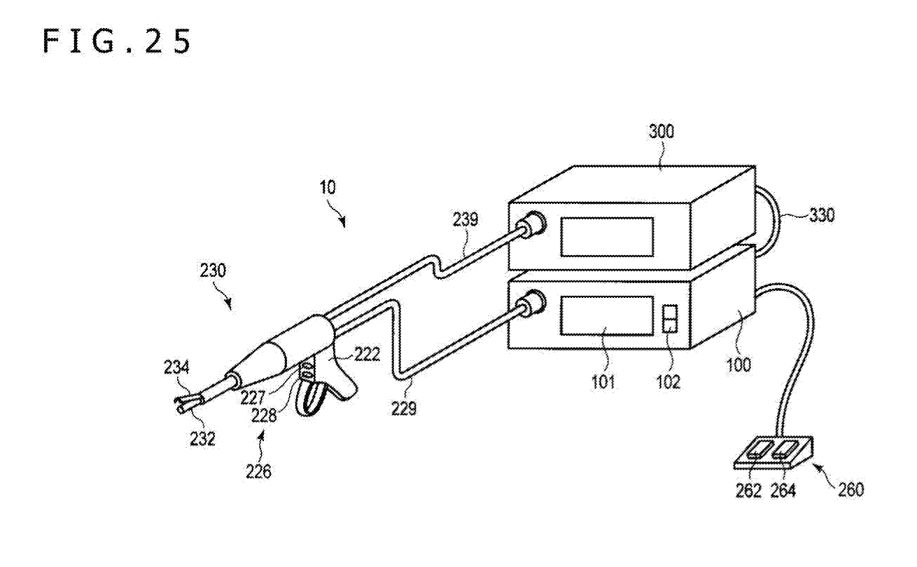

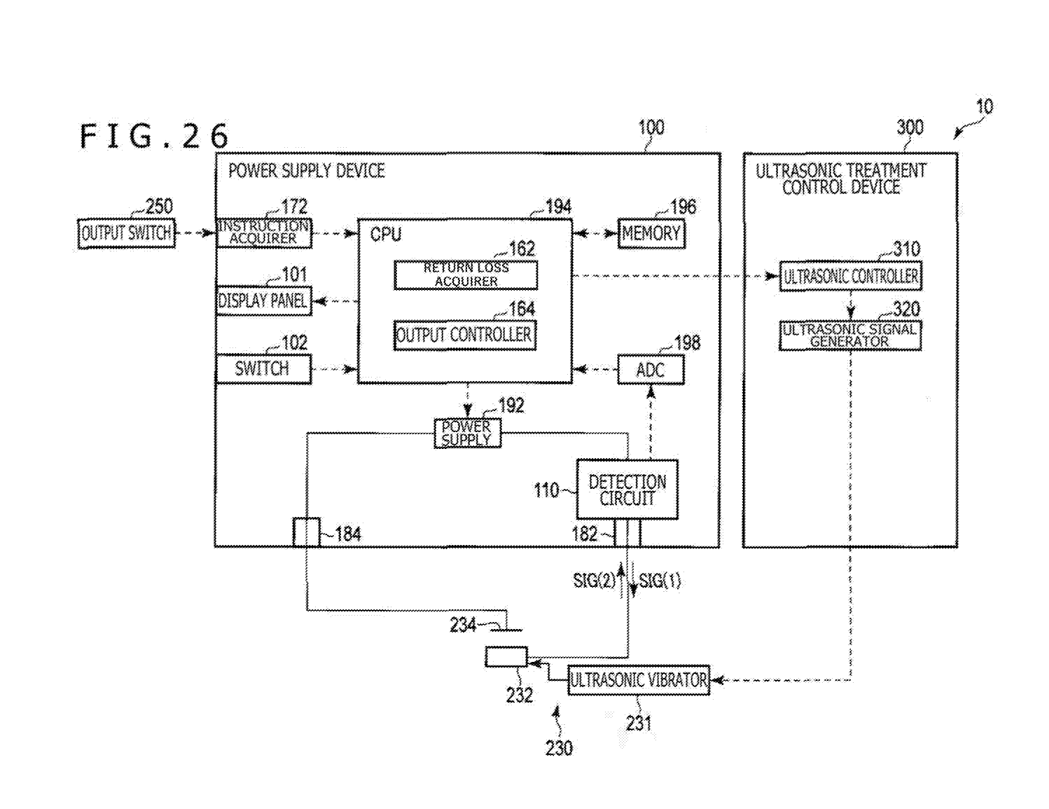

In the embodiment described hereinbefore, furthermore, the treatment tool 220 is illustrated as an instrument for performing only treatments with high-frequency electric power. However, the treatment tool 220 is not limited to such an instrument. The treatment tool may be a treatment tool having an ultrasonically vibratable probe for treating a treatment target using both high-frequency energy and ultrasonic energy. A high-frequency/ultrasonic treatment system 10 using both high-frequency energy and ultrasonic energy according to a modification will be described hereinafter with reference to FIGS. 25 and 26. Features different from the embodiments described hereinbefore will be described hereinafter, and those parts which are identical to those described hereinbefore will be denoted by identical numerical references and will not be described in detail hereinafter to avoid redundancy.

FIG. 25 depicts a general appearance of the high-frequency/ultrasonic treatment system 10 according to the present modification. FIG. 26 illustrates a general configuration of the high-frequency/ultrasonic treatment system 10 according to the present modification. The high-frequency/ultrasonic treatment system 10 includes a high-frequency/ultrasonic treatment tool 230 in place of the treatment tool 220 according to the embodiments described hereinbefore. The high-frequency/ultrasonic treatment tool 230 is a bipolar treatment tool. The high-frequency/ultrasonic treatment tool 230 includes a first electrode 232 corresponding to the distal-end electrode 224 according to the embodiments described hereinbefore. Furthermore, the high-frequency/ultrasonic treatment tool 230 includes a second electrode 234 corresponding to the counter electrode plate 240. Moreover, the high-frequency/ultrasonic treatment tool 230 includes an ultrasonic vibrator 231 that is a vibration source for ultrasonically vibrating the first electrode 232. The first electrode 232 functions as an electrode of the high-frequency treatment tool and also functions as a probe of the high-frequency treatment tool. The second electrode 234 functions as a counter electrode that faces the first electrode 232.

A power supply device 100 and an output switch 250 according to the present modification are identical respectively to the power supply device 100 and the output switch 250 according to the embodiments described hereinbefore. According to the present modification, the high-frequency/ultrasonic treatment system 10 includes, in addition to the power supply device 100, an ultrasonic treatment control device 300 for controlling operation of the ultrasonic vibrator 231. The ultrasonic treatment control device 300 may be included in the power supply device 100.

The ultrasonic treatment control device 300 is connected to the power supply device 100 by a cable 330. The ultrasonic treatment control device 300 is connected to the high-frequency/ultrasonic treatment tool 230 by a cable 239. The ultrasonic treatment control device 300 includes an ultrasonic controller 310 and an ultrasonic signal generator 320. The ultrasonic controller 310 controls operation of various parts of the ultrasonic treatment control device 300 which include the ultrasonic signal generator 320. The CPU 194 is connected to the output controller 164 and the ultrasonic controller 310, and performs processing sequences while grasping the states of those parts. As with the output controller 164, the ultrasonic controller 310 may be included in the CPU 194. The ultrasonic signal generator 320 generates a signal for energizing the ultrasonic vibrator 231 under the control of the ultrasonic controller 310.

For performing a treatment using the high-frequency/ultrasonic treatment tool 230, the user brings the first electrode 232 into contact with the living tissue 900 to be treated, and turns on the output switch 250. At this time, the high-frequency/ultrasonic treatment tool 230 outputs energy. For example, when the first switches 227 and 262 of the output switch 250 are turned on, the ultrasonic controller 310 acquires information indicating that the first switches 227 and 262 are turned on through the output controller 164 and outputs a signal for causing the ultrasonic signal generator 320 to generate ultrasonic energy. Based on the signal thus output, the ultrasonic vibrator 231 vibrates ultrasonically and transmits the ultrasonic vibration to vibrate the first electrode 232 ultrasonically. At the same time, the output controller 164 controls the power supply 192 to output high-frequency electric power. As a result, a high-frequency current flows through the living tissue 900 that is present between the first electrode 232 and the second electrode 234. Heat is generated by friction between the living tissue 900 and the ultrasonically vibrating first electrode 232. The high-frequency current flowing through the living tissue 900 also causes the living tissue 900 to generate heat. These heats treat the living tissue 900 by incising it or stopping bleeding from it.

When the second switches 228 and 264 of the output switch 250 are turned on, only the power supply 192 outputs high-frequency electric power, but the ultrasonic signal generator 320 does not output a signal for generating ultrasonic energy. As a consequence, a high-frequency current flows through the living tissue 900 that is present between the first electrode 232 and the second electrode 234, generating heat. The heat treats the living tissue 900 by stopping bleeding from it, for example.

The ultrasonic vibration energy and the high-frequency electric energy are simultaneously applied through the first electrode 232 to the living tissue 900 to be treated, thereby minimizing sticking of the living tissue to the first electrode 232. As a result, the living tissue 90 is smoothly treated as by being incised or by stopping bleeding therefrom.

Generally, it is understood that when ultrasonic vibrations are applied to the living tissue 900, a small fraction of the living tissue 900 is scattered as a mist. Especially if the living tissue 900 to be treated contains much fat, the fat is scattered as a mist in the middle of the treatment of the living tissue 900. While the mist of scattered fat is drifting around the region being treated, if the first electrode 232 or the second electrode 234 and the living tissue 900 are spaced from each other by a certain distance and the output level of high-frequency electric power is high, an unintended large electric discharge is likely to occur. With the high-frequency/ultrasonic treatment system 10 according to the present modification, as with the embodiments described hereinbefore, a certain state, e.g., the state in which the first electrode 232 or the second electrode 234 and the living tissue 900 are spaced from each other by a certain distance, is detected based on the return loss RL. The output of high-frequency electric power is temporarily reduced in the transition period including such a state. The reduction in the output prevents the output value from instantaneously deviating largely from the target value due to the occurrence of an unintended large electric discharge even in the presence of the suspended mist of fat. The function to temporarily reduce the output of high-frequency electric power is thus particularly effective in the event of a treatment with ultrasonic vibrations as well as high-frequency electric power.

In sum, the technology disclosed herein is directed to a power supply device used in a treatment system which comprises a power supply that supplies high-frequency electric power to an electrode. A detection circuit detects a first signal and a second signal. The first signal is a signal representing an output voltage output from the power supply to a high-frequency treatment tool. The second signal is a signal representing electric power output from the power supply to the high-frequency treatment tool and returned from the high-frequency treatment tool back to the power supply. A controller includes one or more processors as hardware. The one or more processors calculate a return loss representing a state of contact between a living tissue and the electrode based on the first signal and the second signal. The one or more processors then control the power supply to change its output between a first output level which is an output level thereof for treating the living tissue and a second output level which is an output level thereof lower than the first output level, and set the output of the power supply to the second output level when the return loss meets a predetermined switching condition.

Moreover, the technology disclosed herein is directed to a high-frequency treatment system which comprises a power supply device and a high-frequency treatment tool. The power supply device includes a power supply that supplies high-frequency electric power to an electrode. A detection circuit detects a first signal and a second signal. The first signal is a signal representing an output voltage output from the power supply to the high-frequency treatment tool. The second signal is a signal representing electric power output from the power supply to the high-frequency treatment tool and returned from the high-frequency treatment tool back to the power supply. A controller includes one or more processors as hardware. The one or more processors calculate a return loss representing a state of contact between a living tissue and the electrode based on the first signal and the second signal. The one or more processors control the power supply to change its output between a first output level which is an output level thereof for treating the living tissue and a second output level which is an output level thereof lower than the first output level, and set the output of the power supply to the second output level when the return loss meets a predetermined switching condition.

A further aspect of the technology disclosed herein is directed to a method of controlling a high-frequency treatment tool which comprises supplying high-frequency electric power from a power supply to an electrode. Next, the method comprises detecting a first signal and a second signal. The first signal is a signal representing an output voltage output from the power supply to the high-frequency treatment tool. The second signal is a signal representing electric power output from the power supply to the high-frequency treatment tool and returned from the high-frequency treatment tool back to the power supply. Calculating a return loss representing a state of contact between a living tissue and the electrode based on the first signal and the second signal. Finally, controlling the power supply to change its output from a first output level which is an output level thereof for treating the living tissue to a second output level which is an output level thereof lower than the first output level when the return loss meets a predetermined switching condition.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future. The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A power supply device used with a high-frequency treatment tool comprising:
a power supply that supplies high-frequency electric power to an electrode of the high-frequency treatment tool;
a detection circuit that detects a first signal which is a signal representing an output voltage output from the power supply to the high-frequency treatment tool and a second signal which is a signal representing electric power returned from the high-frequency treatment tool back to the power supply; and
a controller;
wherein the controller includes one or more processors as hardware; and
the one or more processors
calculate a return loss representing a state of contact between a living tissue and the electrode based on the first signal and the second signal, and
control the power supply to change its output between a first output level which is an output level thereof for treating the living tissue and a second output level which is an output level thereof lower than the first output level, and set the output of the power supply to the second output level when the return loss meets a predetermined switching condition.

2. The power supply device of claim 1, wherein the high-frequency electric power output from the power supply to the electrode is represented as first electric power, the high-frequency electric power returned from the electrode back to the power supply is represented as second electric power, and the return loss is calculated as a ratio of the second election power to the first electric power.

3. The power supply device of claim 2, further comprising:
a memory;
wherein the memory stores a first return loss when the living tissue and the electrode are spaced from each other by a predetermined distance; and
the controller determines that the switching condition is met when the value of the return loss is smaller than the first return loss stored in the memory.

4. The power supply device of claim 3, wherein the memory stores a maximum value of the return loss; and
the controller determines that the switching condition is met when the difference between the value of the return loss and the maximum value of the return loss is larger than a first threshold value.

5. The power supply device of claim 3, wherein the memory stores a maximum value of the return loss; and
the controller determines that the switching condition is met when the difference between the value of the return loss and the maximum value of the return loss is larger than the first threshold value repeatedly by a predetermined number of times.

6. The power supply device of claim 3, wherein the controller controls the operation to set the output to the first output level upon elapse of a predetermined period after having set the output to the second output level.

7. The power supply device of claim 2, further comprising:
a memory;
wherein the memory stores a first return loss when the living tissue and the electrode are in contact with each other; and
the controller determines that the switching condition is met when the value of the return loss is larger than the first return loss stored in the memory.

8. The power supply device of claim 7, wherein the memory stores a minimum value of the return loss; and
the controller determines that the switching condition is met when the difference between the value of the return loss and the minimum value of the return loss is larger than a first threshold value.

9. The power supply device of claim 7, wherein the memory stores a minimum value of the return loss; and
the controller determines that the switching condition is met when the difference between the value of the return loss and the minimum value of the return loss is larger than the first threshold value repeatedly by a predetermined number of times.

10. The power supply device of claim 1, wherein when the controller changes the output, the controller gradually changes the output.

11. The power supply device of claim 1, wherein a state in which the power supply produces the output thereof at the second output level is a repetition of a state in which the output is set to a third output level lower than the first output level and a state in which the output is set to a fourth output level equal to or lower than the first output level.

12. The power supply device of claim 1, wherein the detection circuit includes a coil and a capacitor.

13. The power supply device of claim 1, wherein the controller switches to the second output level when the return loss is larger than a predetermined first value and smaller than a predetermined second value.

14. A high-frequency treatment system comprising:
a power supply device; and
a high-frequency treatment tool;
wherein the power supply device includes
a power supply that supplies high-frequency electric power to an electrode of the high-frequency treatment tool,
a detection circuit that detects a first signal which is a signal representing an output voltage output from the power supply to the high-frequency treatment tool and a second signal which is a signal representing electric power returned from the high-frequency treatment tool back to the power supply, and
a controller;
the controller includes one or more processors as hardware; and
the one or more processors
calculate a return loss representing a state of contact between a living tissue and the electrode based on the first signal and the second signal, and
control the power supply to change its output between a first output level which is an output level thereof for treating the living tissue and a second output level which is an output level thereof lower than the first output level, and set the output of the power supply to the second output level when the return loss meets a predetermined switching condition.

15. The high-frequency treatment system of claim 14, wherein the high-frequency treatment tool is a high-frequency/ultrasonic treatment tool configured to treat the living tissue also with ultrasonic vibrations.

16. A method of controlling a high-frequency treatment tool of a high-frequency treatment system, comprising:
supplying high-frequency electric power from a power supply of the high-frequency treatment system to an electrode of the high-frequency treatment tool;
detecting a first signal which is a signal representing an output voltage output from the power supply to the high-frequency treatment tool and a second signal which is a signal representing electric power returned from the high-frequency treatment tool back to the power supply;
calculating a return loss representing a state of contact between a living tissue and the electrode based on the first signal and the second signal; and
controlling the power supply to change its output from a first output level which is an output level thereof for treating the living tissue to a second output level which is an output level thereof lower than the first output level when the return loss meets a predetermined switching condition.
